(19) United States
(12) Patent Application Publication
(10) Pub. No.: US 2025/0143795 A1
(43) Pub. Date: May 8, 2025

(54) DYNAMIC FLEXIBLE SCOPE DRIVE AND METHODS OF USING SAME

(71) Applicant: AURIS HEALTH, INC., Santa Clara, CA (US)

(72) Inventors: Drake Michael LONG, Santa Clara, CA (US); Juan Bajana MERIZALDE, San Francisco, CA (US); Fabien Y. SCHMITT, Redwood City, CA (US)

(21) Appl. No.: 19/015,611

(22) Filed: Jan. 9, 2025

Related U.S. Application Data
(63) Continuation of application No. PCT/IB2023/057088, filed on Jul. 11, 2023.
(60) Provisional application No. 63/388,964, filed on Jul. 13, 2022.
(60) Provisional application No. 63/446,308, filed on Feb. 16, 2023.

(51) Int. Cl. A61B 34/00; A61B 1/01; A61B 34/30

(52) U.S. Cl. CPC A61B 34/00; A61B 1/01; A61B 2034/301; A61B 2034/305

(57) ABSTRACT

A surgical robotic system can include a robotic arm coupled to a flexible scope and a viewer for displaying a field of view of a surgical site derived from the flexible scope. The surgical robotic system may be configured to operate the flexible scope in a particular mode of a plurality of modes. In accordance with a determination that the flexible scope is operating in the particular mode, the surgical robotic system may provide electrical signals for presenting a first visual indicator, corresponding to the respective mode, on the viewer.

### PRIORITY

This application is a continuation of International Patent Application No. PCT/IB2023/057088, filed Jul. 11, 2023, entitled “DYNAMIC FLEXIBLE SCOPE DRIVE AND METHODS OF USING SAME,” which claims priority to U.S. Provisional Patent Application No. 63/446,308, entitled “DYNAMIC FLEXIBLE SCOPE DRIVE AND METHODS OF USING SAME,” filed Feb. 16, 2023, and U.S. Provisional Patent Application No. 63/388,964, entitled “DYNAMIC FLEXIBLE SCOPE DRIVE AND METHODS OF USING SAME,” filed Jul. 13, 2022. The contents of all of the above-referenced patent applications are hereby incorporated by reference in their entirety for all purposes.

### TECHNICAL FIELD

The systems and methods disclosed herein are directed to devices and methods for indicating locations or orientations of surgical tools, and more particularly to surgical robotic systems for indicating locations or orientations of flexible surgical tools.

### BACKGROUND

A robotically enabled medical system is capable of performing a variety of medical procedures, including both minimally invasive procedures, such as laparoscopy, and non-invasive procedures, such as endoscopy (e.g., bronchoscopy, ureteroscopy, gastroscopy, etc.).

Such robotic medical systems may include robotic arms configured to control the movement of surgical tool(s) during a given medical procedure. In order to achieve a desired pose of a surgical tool, a robotic arm may be placed into a particular pose during teleoperation. Some robotically enabled medical systems may include an arm support (e.g., a bar) that is connected to respective bases of the robotic arms and supports the robotic arms.

### SUMMARY

One or more instruments can be coupled to one or more robotic arms of a robotic medical system (e.g., surgical robotic system) for medical procedures. For example, an instrument can be coupled to a robotic arm as either a starting instrument to perform a procedure, or as a replacement instrument mid-procedure.

In a robotic system that includes multiple robotic arms, at least one robotic arm can be coupled to a camera or scope that provides a surgical field of view. The scope is a vital asset for laparoscopic or endoscopic surgery. A robotic scope that is straight and rigid can be controlled in a limited number of ways. For example, the scope can be inserted, removed, panned left and right, and rotated. However, a rigid scope can present challenges in a robotic system that includes multiple robotic arms. For example, the limited number of ways in which a rigid scope can be controlled can lead to increased collisions between robotic arms. Furthermore, rigid scopes may be large in size, which can crowd a surgeon's workspace or further limit the area where surgical instruments and ports may be located on the patient.

To mitigate these challenges, a robotic arm can be coupled with a flexible scope (e.g., a flexible laparoscope, a flexible endoscope, etc.) that includes a camera. The flexible scope introduces additional degrees of freedom, allowing a surgeon to control the camera coupled thereto in more ways than those available with a rigid scope. For example, in addition to the insertion, removal, pan and rotate operations that are achievable with a rigid scope, a user can also move and rotate (e.g., articulate, pivot, flex, bend, etc.) at least a portion (e.g., a distal portion) of the flexible scope. Consequently, the user can view a target anatomy from perspectives (e.g., angles) that are not achievable with a rigid scope. In some instances, a flexible scope can have a volume that is smaller than that of a rigid scope, thereby freeing up space in the surgeon's workspace. Operating a flexible scope can present new challenges, however. For example, a user may not be able to readily determine the shape of the flexible scope, or a direction and/or extent of movement or rotation of the scope, or how to return the scope from a bent configuration to a straight configuration.

Accordingly, there is a need for a robotic medical system that can facilitate the user to visualize the position or orientation of a flexible scope in connection with a surgical field of view.

As disclosed herein, a robotic medical system (e.g., a surgical robotic system) can include a robotic arm that is coupled to a flexible scope. The robotic medical system also includes a viewer for displaying a field of view of a surgical site derived from the flexible scope. For example, the field of view of the surgical site may correspond to (or may be determined based on) the field of view of a camera coupled to the flexible scope. A user interface displayed on the viewer may be configured to include an indicator, which indicates a position and/or an orientation of the flexible scope relative to a reference position.

As disclosed herein, the flexible scope can be inserted into a patient through a port and the reference position is determined based on (e.g., corresponds to) a position of the port.

As disclosed herein, the robotic medical system is configured to operate the flexible scope in a plurality of modes, such as an articulation mode, an orbit mode, and an automatic insertion and/or retraction mode (also called an automatic insertion-retraction mode).

As disclosed herein, the robotic medical system has knowledge of the camera field of view (e.g., by storing information indicating the camera field of view). In some embodiments, the field of view is determined based on an image provided by the camera. In some embodiments, the field of view is determined based on a position and an orientation of the camera (e.g., a predefined volume of space in front of the camera).

As disclosed herein, the robotic medical system is configured to allow movement of the robotic arms. In some embodiments, the robotic medical system may cause robotic movement of the robotic arms. In some embodiments, the robotic medical system may allow manual movement of the robotic arms.

As disclosed herein, the robotic medical system is configured to determine a position and/or orientation of the flexible scope. In some embodiments, the robotic medical system determines that a position or an orientation of the scope has changed and updates the indicator to indicate the changed position or orientation of the scope. In some embodiments, the robotic medical system updates the indicator in real time (e.g., while the scope is moving) to indicate the changed position or orientation of the scope.

Accordingly, the systems and/or methods disclosed herein advantageously improve the operation of robotic medical systems during surgery. For example, the use of a flexible scope enables a user to view a target anatomy at perspectives that may not be achievable with a rigid scope. The user interface displayed on the viewer may be configured to include an indicator of a position or an orientation of the flexible scope, which leads to enhanced user experience because the user can determine the position and/or orientation of the flexible scope quickly. The ability to determine the position or orientation of the scope may also improve the safety of the surgery.

The systems, methods, and devices of this disclosure each have several innovative aspects, no single one of which is solely responsible for the desirable attributes disclosed herein.

In accordance with some embodiments of the present disclosure, a robotic system includes a first robotic arm coupled to a flexible scope. The robotic system also includes a viewer for displaying a field of view of a surgical site derived from the flexible scope. The robotic system further includes one or more processors, and memory storing instructions for execution by the one or more processors. The stored instructions include instructions for operating the flexible scope in a particular mode of a plurality of modes. The stored instructions include instructions for, in accordance with a determination that the flexible scope is operating in the particular mode of the plurality of modes, provide electrical signals for presenting a first visual indicator, corresponding to the respective mode, on the viewer. The first visual indicator indicates at least one of: a position or an orientation of the flexible scope relative to a reference position.

In some embodiments, the flexible scope is inserted into a patient through a first port and the reference position is determined based on a position of the first port.

In some embodiments, the robotic system causes movement of a distal portion of the flexible scope from a first position or a first orientation to a second position distinct from the first position or a second orientation distinct from the first orientation while the robotic system is operating the flexible scope in a first mode of the plurality of modes.

In some embodiments, the stored instructions include instructions for updating the first visual indicator to indicate the second position or the second orientation of the distal portion of the flexible scope relative to the reference position.

In some embodiments, the first visual indicator includes one or more reference axes and a graphical element positioned relative to the one or more reference axes to identify at least one of a direction or an extent of the movement.

In some embodiments, the stored instructions also include instructions for, in accordance with a determination that the distal portion of the flexible scope has moved from at least one of: (i) the first position to the second position or (ii) the first orientation to the second orientation, providing electrical signals for presenting an updated graphical element that indicates a change in the position and/or orientation of the flexible scope associated with the movement.

In some embodiments, the stored instructions also include instructions for determining a shape of the flexible scope and providing an image that corresponds to the determined shape.

In some embodiments, the stored instructions also include instructions for, in accordance with a determination that the shape of the flexible scope has changed from a first shape to a second shape, providing electrical signals for presenting an updated image that indicates the changed shape.

In some embodiments, the image includes two or more links that correspond to two or more links of the flexible scope.

In some embodiments, the first visual indicator includes a plurality of graphical elements. The plurality of graphical elements includes a first graphical element and a second graphical element distinct from the first graphical element. The first graphical element represents a first component of the movement of the distal end of the flexible scope along a first direction. The second graphical element represents a second component of the movement of the distal end of the flexible scope along a second direction, wherein the first direction is distinct from the second direction.

In some embodiments, the stored instructions include instructions for, in accordance with a determination that the movement of the distal end of the flexible scope along the first direction has changed from a first magnitude to a second magnitude, providing electrical signals for updating a location of the first graphical element. The stored instructions also include instructions for, in accordance with a determination that the movement of the distal end of the flexible scope along the second direction has changed from a third magnitude to a fourth magnitude, providing electrical signals for updating a location of the second graphical element.

In some embodiments, the stored instructions include instructions for, in accordance with a determination that the movement of the flexible scope in the first direction has changed from a first magnitude to a second magnitude, providing electrical signals for updating a length of the first graphical element. The stored instructions also include instructions for, in accordance with a determination that the movement of the flexible scope in the second direction has changed from a third magnitude to a fourth magnitude, providing electrical signals for updating a length of the second graphical element.

In some embodiments, the field of view of the surgical site is derived from a camera coupled to the flexible scope. The stored instructions also include instructions for expanding the first visual indicator in accordance with a determination that the camera is active.

In some embodiments, the robotic system causes movement of the flexible scope about a target point in space while the robotic system is operating the flexible scope in a second mode of the plurality of modes.

In some embodiments, the first visual indicator comprises a first graphical element representing the target point.

In some embodiments, the stored instructions also include instructions for providing electrical signals for overlaying the first graphical element on the displayed field of view.

In some embodiments, the first visual indicator includes a second graphical element representing a line of sight from the target point to a port of the flexible scope.

In some embodiments, the first graphical element has a first size. The stored instructions also include instructions for adjusting a size of the first graphical element from the first size to a second size according to a change in a depth of insertion of the flexible scope into the surgical site.

In some embodiments, the first visual indicator includes a second graphical element representing a range of orbit motion of the flexible scope.

In some embodiments, the first visual indicator includes a third graphical element representing a position of the flexible scope.

In some embodiments, the stored instructions also include instructions for providing electrical signals for overlaying the first graphical element, the second graphical element, and the third graphical element on the displayed field of view.

In some embodiments, the first visual indicator includes one or more axes and a graphical element that represents an insertion direction of the flexible scope relative to the one or more axes.

In some embodiments, the robotic system causes the flexible scope to automatically insert and/or retract when the robotic system is operating the flexible scope in a third mode of the plurality of modes.

In some embodiments, the stored instructions also include instructions for determining a shape of the flexible scope during activation of the third mode; and providing an image that corresponds to the determined shape.

In some embodiments, the stored instructions also include instructions for updating the image while the flexible scope is automatically inserted or retracted.

In some embodiments, the first visual indicator includes a graphical element that indicates at least one of: an extent of insertion, an extent of retraction, or an articulation of the flexible scope.

In some embodiments, the robotic system includes a second robotic arm coupled to a surgical tool.

In some embodiments, the stored instructions include instructions for providing electrical signals for presenting a second visual indicator, corresponding to the surgical tool, on the viewer.

In some embodiments, the first visual indicator includes information identifying the first robotic arm coupled to the flexible scope.

In some embodiments, the first visual indicator is concurrently displayed with a display of the field of view of the surgical site on the viewer.

In some embodiments, the first visual indicator is displayed around the display of the field of view of the surgical site.

In some embodiments, the first visual indicator is displayed over the display of the field of view of the surgical site.

In some embodiments, the viewer is part of a surgeon console.

In some embodiments, the surgeon console includes an input device. The stored instructions include instructions for receiving an input on the input device and determining that the flexible scope is operating in the particular mode in accordance with the received input. In some embodiments, the input device includes a foot pedal.

In accordance with some embodiments of the present disclosure, a robotic system includes a first robotic arm coupled to a flexible scope. The robotic system includes a viewer for displaying a field of view of a surgical site derived from the flexible scope. The robotic system also includes one or more processors and memory storing instructions for execution by the one or more processors. The stored instructions include instructions for operating the flexible scope and providing electrical signals for presenting a first visual indicator on the viewer. The first visual indicator indicates at least one of: a position or an orientation of the flexible scope relative to a reference position.

In accordance with some embodiments, an electronic device is in communication with a robotic system having a first robotic arm coupled to a flexible scope and a viewer for displaying a field of view of a surgical site derived from the flexible scope. The electronic device includes one or more processors and memory. The memory stores instructions for execution by the one or more processors. The stored instructions include instructions for operating the flexible scope in a particular mode of a plurality of modes. The stored instructions also include instructions for, in accordance with a determination that the flexible scope is operating in the particular mode of the plurality of modes, providing electrical signals for presenting a first visual indicator, corresponding to the respective mode, on the viewer. The first visual indicator indicates at least one of: a position or an orientation of the flexible scope relative to a reference position.

In accordance with some embodiments, an electronic device is in communication with a robotic system having a first robotic arm coupled to a flexible scope and a viewer for displaying a field of view of a surgical site derived from the flexible scope. The electronic device includes one or more processors and memory. The memory stores instructions for execution by the one or more processors. The stored instructions include instructions for operating the flexible scope; and providing electrical signals for presenting a first visual indicator on the viewer. The first visual indicator indicates at least one of: a position or an orientation of the flexible scope relative to a reference position.

In accordance with some embodiments, a computer-readable storage medium stores instructions for execution by one or more processors in communication with a robotic system with a first robotic arm coupled to a flexible scope and a viewer for displaying a field of view of a surgical site derived from the flexible scope. The stored instructions include instructions for operating the flexible scope in a particular mode of a plurality of mode. The stored instructions include instructions for, in accordance with a determination that the flexible scope is operating in the particular mode of the plurality of modes, providing electrical signals for presenting a first visual indicator, corresponding to the respective mode, on the viewer. The first visual indicator indicates at least one of: a position or an orientation of the flexible scope relative to a reference position.

In accordance with some embodiments, a computer-readable storage medium stores instructions for execution by one or more processors in communication with a robotic system with a first robotic arm coupled to a flexible scope and a viewer for displaying a field of view of a surgical site derived from the flexible scope. The stored instructions include instructions for operating the flexible scope in a particular mode of a plurality of mode. The stored instructions include instructions for operating the flexible scope; and providing electrical signals for presenting a first visual indicator on the viewer. The first visual indicator indicates at least one of: a position or an orientation of the flexible scope relative to a reference position.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy procedure(s).

FIG. 2 depicts further aspects of the robotic system of FIG. 1.

FIG. 3 illustrates an embodiment of the robotic system of FIG. 1 arranged for ureteroscopy.

FIG. 4 illustrates an embodiment of the robotic system of FIG. 1 arranged for a vascular procedure.

FIG. 5 illustrates an embodiment of a table-based robotic system arranged for a bronchoscopy procedure.

FIG. 6 provides an alternative view of the robotic system of FIG. 5.

FIG. 7 illustrates an example system configured to stow robotic arm(s).

FIG. 8 illustrates an embodiment of a table-based robotic system configured for a ureteroscopy procedure.

FIG. 9 illustrates an embodiment of a table-based robotic system configured for a laparoscopic procedure.

FIG. 10 illustrates an embodiment of the table-based robotic system of FIGS. 5-9 with pitch or tilt adjustment.

FIG. 11 provides a detailed illustration of the interface between the table and the column of the table-based robotic system of FIGS. 5-10.

FIG. 12 illustrates an alternative embodiment of a table-based robotic system.

FIG. 13 illustrates an end view of the table-based robotic system of FIG. 12.

FIG. 14 illustrates an end view of a table-based robotic system with robotic arms attached thereto.

FIG. 15 illustrates an exemplary instrument driver.

FIG. 16 illustrates an exemplary medical instrument with a paired instrument driver.

FIG. 17 illustrates an alternative design for an instrument driver and instrument where the axes of the drive units are parallel to the axis of the elongated shaft of the instrument.

FIG. 18 illustrates an instrument having an instrument-based insertion architecture.

FIG. 19 illustrates an exemplary controller.

FIG. 20 depicts a block diagram illustrating a localization system that estimates a location of one or more elements of the robotic systems of FIGS. 1-10, such as the location of the instrument of FIGS. 16-18, in accordance with an example embodiment.

FIG. 21 illustrates an exemplary robotic system according to some embodiments.

FIG. 22 illustrates another view of an exemplary robotic system according to some embodiments.

FIG. 23 illustrates components of a robotic medical system in accordance with some embodiments.

FIGS. 24A, 24B, and 24C illustrate different views of an exemplary robotic arm according to some embodiments.

FIG. 25 illustrates a perspective view of a robotic medical system in accordance with some embodiments.

FIGS. 26A and 26B illustrate a camera field of view in a three-dimensional space in accordance with some embodiments.

FIGS. 27A, 27B, 27C, and 27D illustrate a flexible scope in accordance with some embodiments.

FIGS. 28A, 28B, 28C, and 28D illustrate exemplary user interfaces displayed on a display device when the flexible scope is operating in an articulation drive mode, in accordance with some embodiments.

FIGS. 29A, 29B, 29C, and 29D illustrate exemplary user interfaces displayed on a display device when the flexible scope is operating in an orbit drive mode, in accordance with some embodiments.

FIGS. 30A, 30B, 30C, 30D, 30E, and 30F illustrate exemplary user interfaces displayed on a display device when the flexible scope is operating in an automated insertion and retraction mode, in accordance with some embodiments.

FIG. 31A illustrates different configurations of flexible scopes in accordance with some embodiments.

FIGS. 31B-31F illustrate exemplary user interface elements displayed on a display device to indicate a status of the flexible scope in accordance with some embodiments.

FIGS. 32A, 32B, 32C, 32D, 32E, and 32F illustrate a flowchart diagram for a method performed by one or more processors of a robotic system, in accordance with some embodiments.

FIG. 33 is a schematic diagram illustrating electronic components of a robotic medical system in accordance with some embodiments.

### DETAILED DESCRIPTION

### 1. Overview.

Aspects of the present disclosure may be integrated into a robotically enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other embodiments of the disclosed concepts are possible, and various advantages can be achieved with the disclosed embodiments. Headings are included herein for reference and to aid in locating various sections. These headings are not intended to limit the scope of the concepts described with respect thereto. Such concepts may have applicability throughout the entire specification.

### A. Robotic System—Cart.

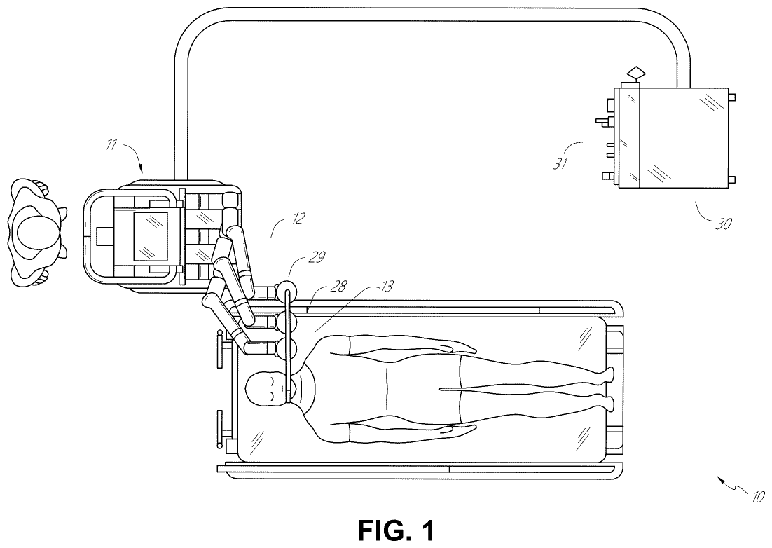

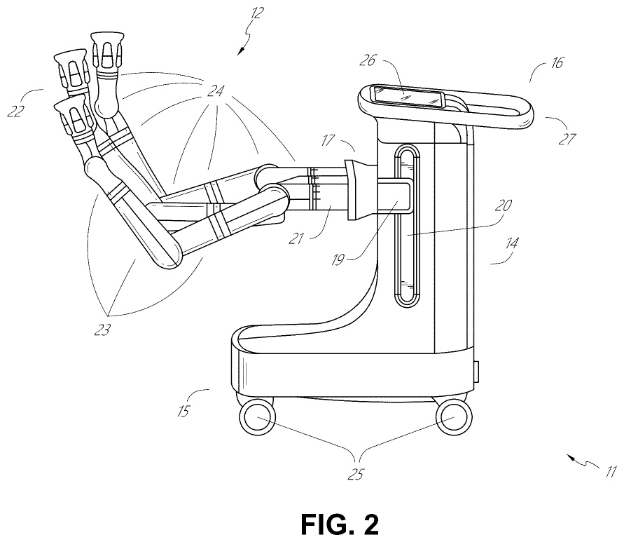

The robotically enabled medical system may be configured in a variety of ways depending on the particular procedure. FIG. 1 illustrates an embodiment of a cart-based robotically enabled system 10 arranged for a diagnostic and/or therapeutic bronchoscopy procedure. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures. FIG. 2 depicts an example embodiment of the cart in greater detail.

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independent of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to resect the potentially cancerous tissue. In some instances, diagnostic and therapeutic treatments can be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or re-positioned by an operating physician and his/her staff. Additionally, the division of functionality between the cart/table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of tower 30. In some embodiments, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include opto-electronics equipment for detecting, receiving, and processing data received from the optical sensors or cameras throughout the robotic system 10. In combination with the control system, such opto-electronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in system 10 are generally designed to provide both robotic controls as well as pre-operative and real-time information of the procedure, such as navigational and localization information of the endoscope 13. When the console 31 is not the only console available to the physician, it may be used by a second operator, such as a nurse, to monitor the health or vitals of the patient and the operation of system, as well as provide procedure-specific data, such as navigational and localization information. In other embodiments, the console 30 is housed in a body that is separate from the tower 30.

The tower 30 may be coupled to the cart 11 and endoscope 13 through one or more cables or connections (not shown). In some embodiments, the support functionality from the tower 30 may be provided through a single cable to the cart 11, simplifying and de-cluttering the operating room. In other embodiments, specific functionality may be coupled in separate cabling and connections. For example, while power may be provided through a single power cable to the cart, the support for controls, optics, fluidics, and/or navigation may be provided through a separate cable.

FIG. 2 provides a detailed illustration of an embodiment of the cart from the cart-based robotically enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of robotic arms 12 to be angled in a variety of configurations.

In some embodiments, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm. Each of the arms 12 have seven joints, and thus provide seven degrees of freedom. A multitude of joints result in a multitude of degrees of freedom, allowing for “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and arms 12 over the floor. Accordingly, the cart base 15 houses heavier components, such as electronics, motors, power supply, as well as components that either enable movement and/or immobilize the cart. For example, the cart base 15 includes rollable wheel-shaped casters 25 that allow for the cart to easily move around the room prior to a procedure. After reaching the appropriate position, the casters 25 may be immobilized using wheel locks to hold the cart 11 in place during the procedure.

Positioned at the vertical end of column 14, the console 16 allows for both a user interface for receiving user input and a display screen (or a dual-purpose device such as, for example, a touchscreen 26) to provide the physician user with both pre-operative and intra-operative data. Potential pre-operative data on the touchscreen 26 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 16 may be positioned and tilted to allow a physician to access the console from the side of the column 14 opposite carriage 17. From this position, the physician may view the console 16, robotic arms 12, and patient while operating the console 16 from behind the cart 11. As shown, the console 16 also includes a handle 27 to assist with maneuvering and stabilizing cart 11.

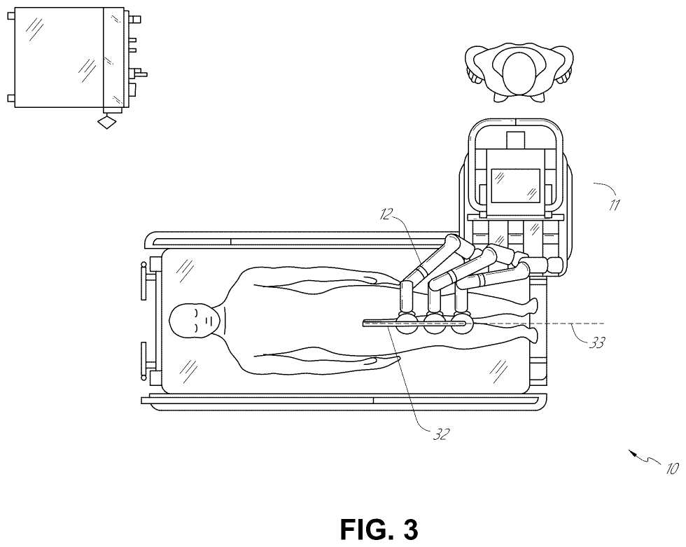

FIG. 3 illustrates an embodiment of a robotically enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

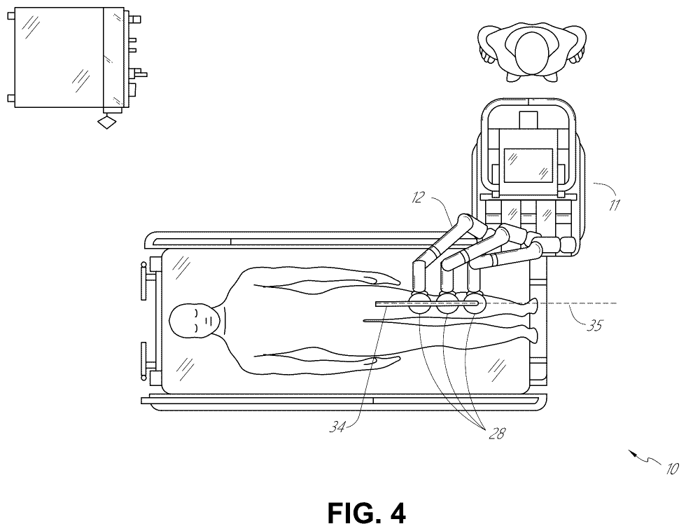

FIG. 4 illustrates an embodiment of a robotically enabled system similarly arranged for a vascular procedure. In a vascular procedure, the system 10 may be configured such that the cart 11 may deliver a medical instrument 34, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

### B. Robotic System—Table.

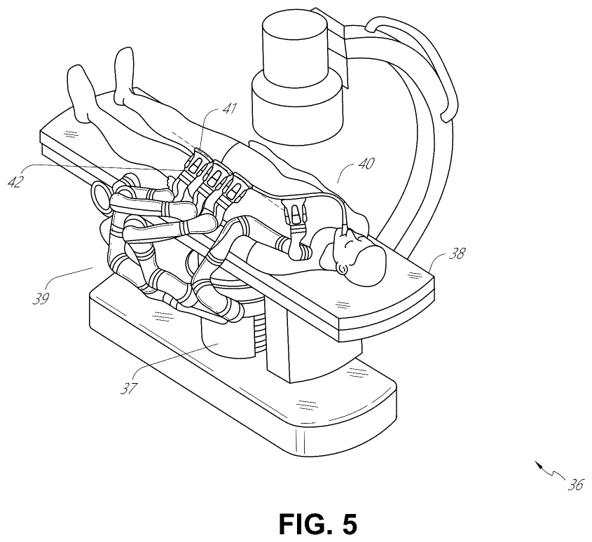

Embodiments of the robotically enabled medical system may also incorporate the patient's table. Incorporation of the table reduces the amount of capital equipment within the operating room by removing the cart, which allows greater access to the patient. FIG. 5 illustrates an embodiment of such a robotically enabled system arranged for a bronchoscopy procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around table 38.

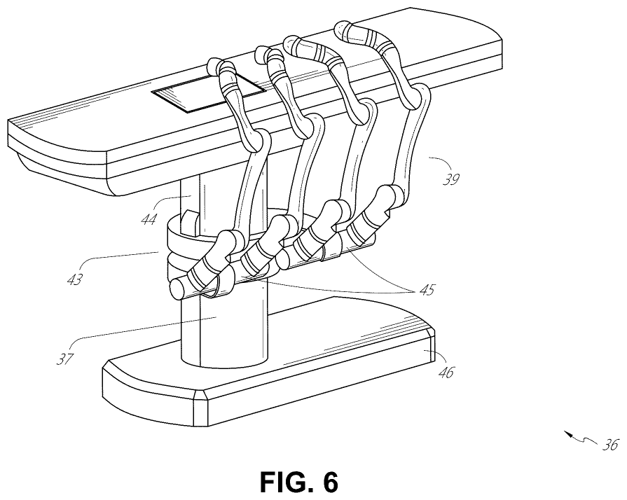

FIG. 6 provides an alternative view of the system 36 without the patient and medical instrument for discussion purposes. As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient. In embodiments with multiple carriages, the carriages may be individually positioned on the column and may translate and/or rotate independent of the other carriages. While carriages 43 need not surround the column 37 or even be circular, the ring-shape as shown facilitates rotation of the carriages 43 around the column 37 while maintaining structural balance. Rotation and translation of the carriages 43 allows the system to align the medical instruments, such as endoscopes and laparoscopes, into different access points on the patient. In other embodiments (not shown), the system 36 can include a patient table or bed with adjustable arm supports in the form of bars or rails extending alongside it. One or more robotic arms 39 (e.g., via a shoulder with an elbow joint) can be attached to the adjustable arm supports, which can be vertically adjusted. By providing vertical adjustment, the robotic arms 39 are advantageously capable of being stowed compactly beneath the patient table or bed, and subsequently raised during a procedure.

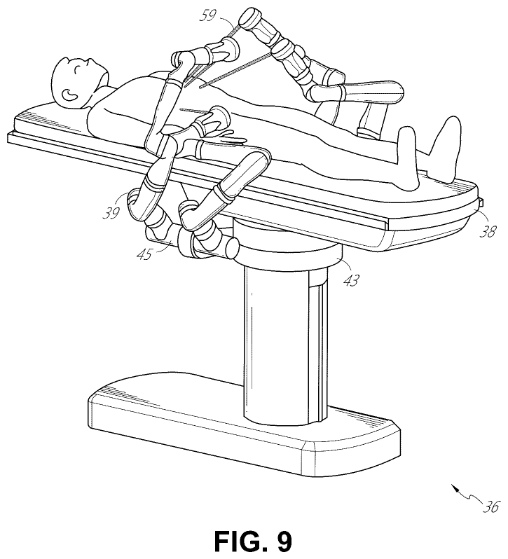

The arms 39 may be mounted on the carriages through a set of arm mounts 45 comprising a series of joints that may individually rotate and/or telescopically extend to provide additional configurability to the robotic arms 39. Additionally, the arm mounts 45 may be positioned on the carriages 43 such that, when the carriages 43 are appropriately rotated, the arm mounts 45 may be positioned on either the same side of table 38 (as shown in FIG. 6), on opposite sides of table 38 (as shown in FIG. 9), or on adjacent sides of the table 38 (not shown).

The column 37 structurally provides support for the table 38, and a path for vertical translation of the carriages. Internally, the column 37 may be equipped with lead screws for guiding vertical translation of the carriages, and motors to mechanize the translation of said carriages based the lead screws. The column 37 may also convey power and control signals to the carriage 43 and robotic arms 39 mounted thereon.

The table base 46 serves a similar function as the cart base 15 in cart 11 shown in FIG. 2, housing heavier components to balance the table/bed 38, the column 37, the carriages 43, and the robotic arms 39. The table base 46 may also incorporate rigid casters to provide stability during procedures. Deployed from the bottom of the table base 46, the casters may extend in opposite directions on both sides of the base 46 and retract when the system 36 needs to be moved.

Continuing with FIG. 6, the system 36 may also include a tower (not shown) that divides the functionality of system 36 between table and tower to reduce the form factor and bulk of the table. As in earlier disclosed embodiments, the tower may provide a variety of support functionalities to table, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base for potential stowage of the robotic arms. The tower may also include a master controller or console that provides both a user interface for user input, such as keyboard and/or pendant, as well as a display screen (or touchscreen) for pre-operative and intra-operative information, such as real-time imaging, navigation, and tracking information. In some embodiments, the tower may also contain holders for gas tanks to be used for insufflation.

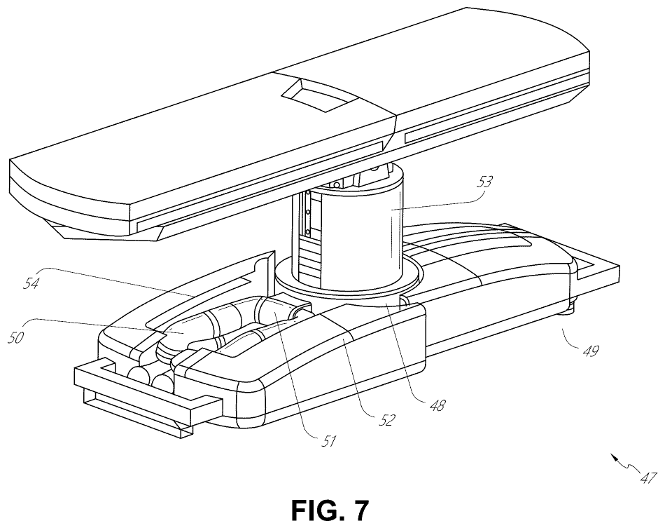

In some embodiments, a table base may stow and store the robotic arms when not in use. FIG. 7 illustrates a system 47 that stows robotic arms in an embodiment of the table-based system. In system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and arms 50 around column 53, and closed to stow to protect them when not in use. The base covers 52 may be sealed with a membrane 54 along the edges of its opening to prevent dirt and fluid ingress when closed.

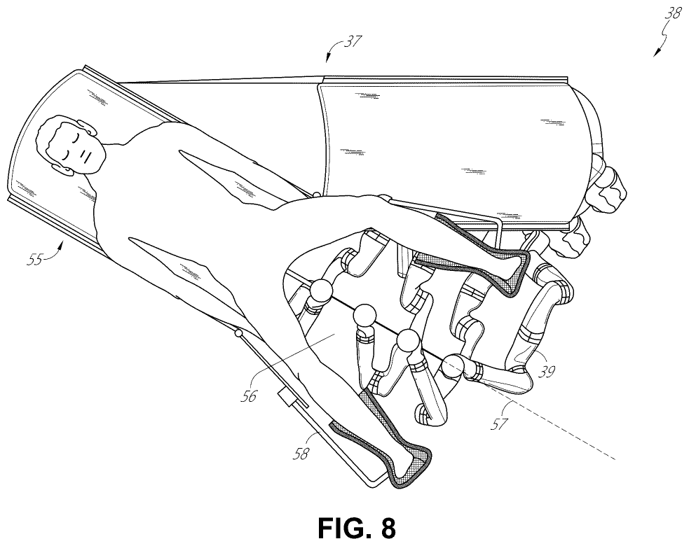

FIG. 8 illustrates an embodiment of a robotically enabled table-based system configured for a ureteroscopy procedure. In a ureteroscopy, the table 38 may include a swivel portion 55 for positioning a patient off-angle from the column 37 and table base 46. The swivel portion 55 may rotate or pivot around a pivot point (e.g., located below the patient's head) in order to position the bottom portion of the swivel portion 55 away from the column 37. For example, the pivoting of the swivel portion 55 allows a C-arm (not shown) to be positioned over the patient's lower abdomen without competing for space with the column (not shown) below table 38. By rotating the carriage 35 (not shown) around the column 37, the robotic arms 39 may directly insert a ureteroscope 56 along a virtual rail 57 into the patient's groin area to reach the urethra. In a ureteroscopy, stirrups 58 may also be fixed to the swivel portion 55 of the table 38 to support the position of the patient's legs during the procedure and allow clear access to the patient's groin area.

In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. FIG. 9 illustrates an embodiment of a robotically enabled table-based system configured for a laparoscopic procedure. As shown in FIG. 9, the carriages 43 of the system 36 may be rotated and vertically adjusted to position pairs of the robotic arms 39 on opposite sides of the table 38, such that instrument 59 may be positioned using the arm mounts 45 to be passed through minimal incisions on both sides of the patient to reach his/her abdominal cavity.

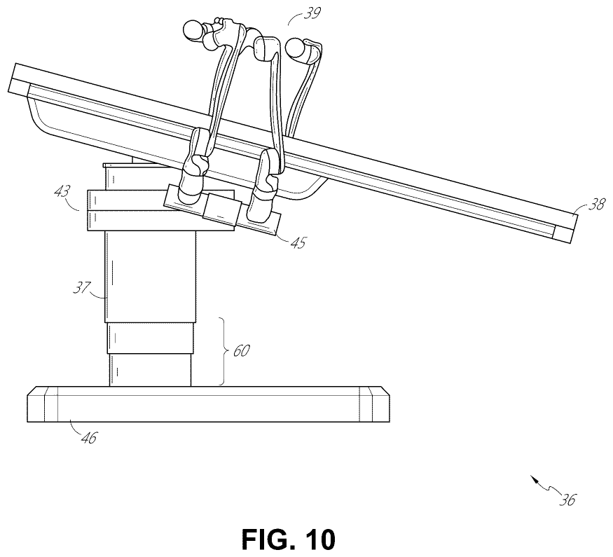

To accommodate laparoscopic procedures, the robotically enabled table system may also tilt the platform to a desired angle. FIG. 10 illustrates an embodiment of the robotically enabled medical system with pitch or tilt adjustment. As shown in FIG. 10, the system 36 may accommodate tilt of the table 38 to position one portion of the table at a greater distance from the floor than the other. Additionally, the arm mounts 45 may rotate to match the tilt such that the arms 39 maintain the same planar relationship with table 38. To accommodate steeper angles, the column 37 may also include telescoping portions 60 that allow vertical extension of column 37 to keep the table 38 from touching the floor or colliding with base 46.

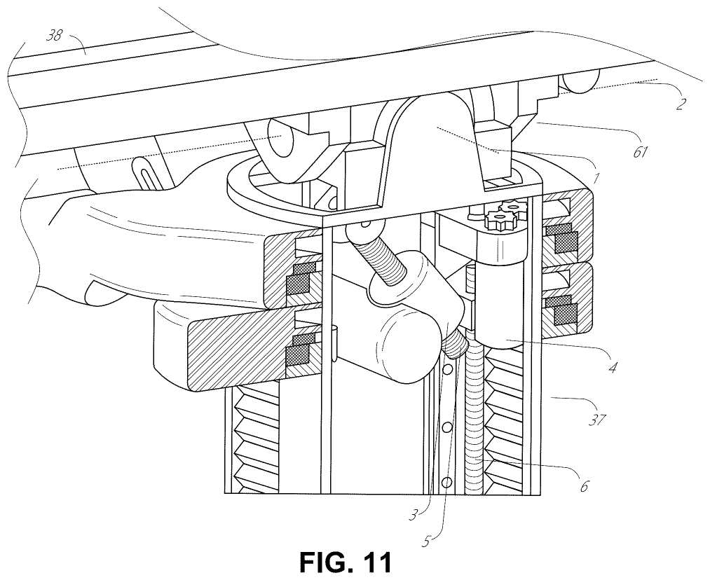

FIG. 11 provides a detailed illustration of the interface between the table 38 and the column 37. Pitch rotation mechanism 61 may be configured to alter the pitch angle of the table 38 relative to the column 37 in multiple degrees of freedom. The pitch rotation mechanism 61 may be enabled by the positioning of orthogonal axes 1, 2 at the column-table interface, each axis actuated by a separate motor 3, 4 responsive to an electrical pitch angle command. Rotation along one screw 5 would enable tilt adjustments in one axis 1, while rotation along the other screw 6 would enable tilt adjustments along the other axis 2. In some embodiments, a ball joint can be used to alter the pitch angle of the table 38 relative to the column 37 in multiple degrees of freedom.

For example, pitch adjustments are particularly useful when trying to position the table in a Trendelenburg position, i.e., position the patient's lower abdomen at a higher position from the floor than the patient's lower abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

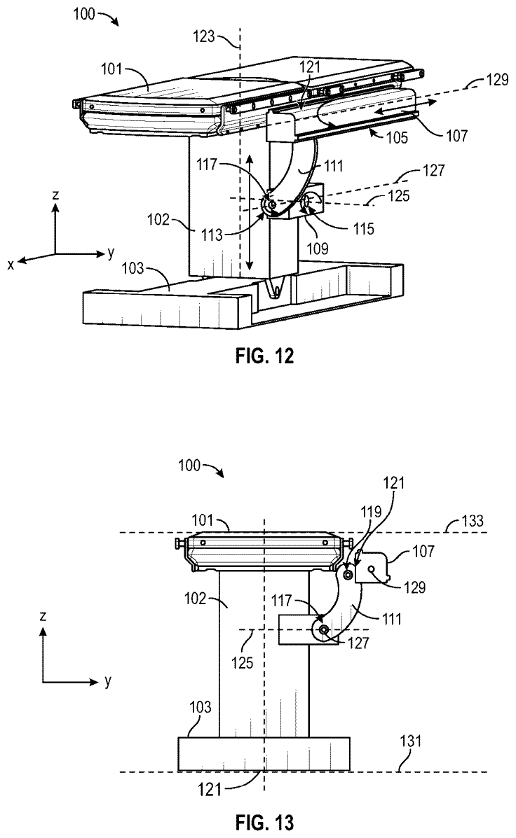

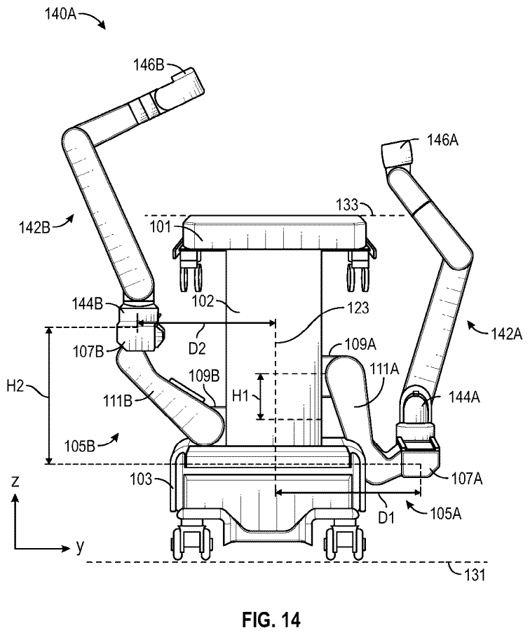

FIGS. 12 and 13 illustrate isometric and end views of an alternative embodiment of a table-based surgical robotics system 100. The surgical robotics system 100 includes one or more adjustable arm supports 105 that can be configured to support one or more robotic arms (see, for example, FIG. 14) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 12 and 13, the arm support 105 is configured with four degrees of freedom, which are illustrated with arrows in FIG. 12. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 12 and 13 can comprise a table supported by a column 102 that is mounted to a base 103. The base 103 and the column 102 support the table 101 relative to a support surface. A floor axis 131 and a support axis 133 are shown in FIG. 13.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

The carriage 109 can be attached to the column 102 by a first joint 113, which allows the carriage 109 to move relative to the column 102 (e.g., such as up and down a first or vertical axis 123). The first joint 113 can provide the first degree of freedom (“Z-lift”) to the adjustable arm support 105. The adjustable arm support 105 can include a second joint 115, which provides the second degree of freedom (tilt) for the adjustable arm support 105. The adjustable arm support 105 can include a third joint 117, which can provide the third degree of freedom (“pivot up”) for the adjustable arm support 105. An additional joint 119 (shown in FIG. 13) can be provided that mechanically constrains the third joint 117 to maintain an orientation of the rail 107 as the rail connector 111 is rotated about a third axis 127. The adjustable arm support 105 can include a fourth joint 121, which can provide a fourth degree of freedom (translation) for the adjustable arm support 105 along a fourth axis 129.

FIG. 14 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

### C. Instrument Driver & Interface.

The end effectors of the system's robotic arms comprise (i) an instrument driver (alternatively referred to as “instrument drive mechanism” or “instrument device manipulator”) that incorporate electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

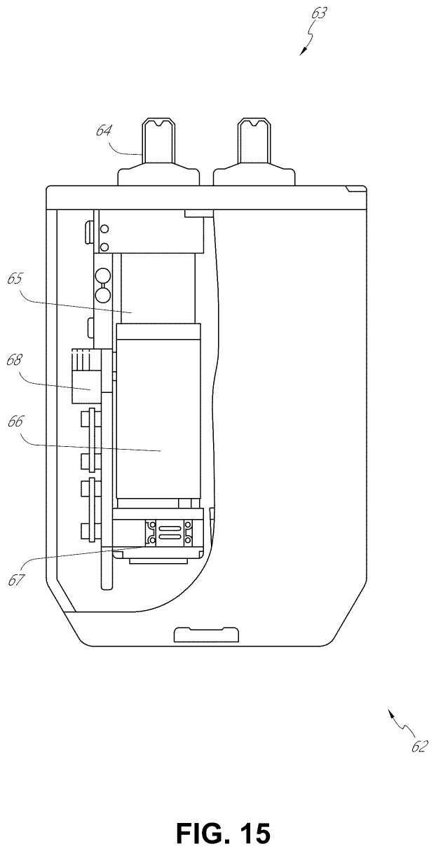

FIG. 15 illustrates an example instrument driver. Positioned at the distal end of a robotic arm, instrument driver 62 comprises of one or more drive units 63 arranged with parallel axes to provide controlled torque to a medical instrument via drive shafts 64. Each drive unit 63 comprises an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuitry 68 for receiving control signals and actuating the drive unit. Each drive unit 63 being independent controlled and motorized, the instrument driver 62 may provide multiple (four as shown in FIG. 15) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise of a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument.

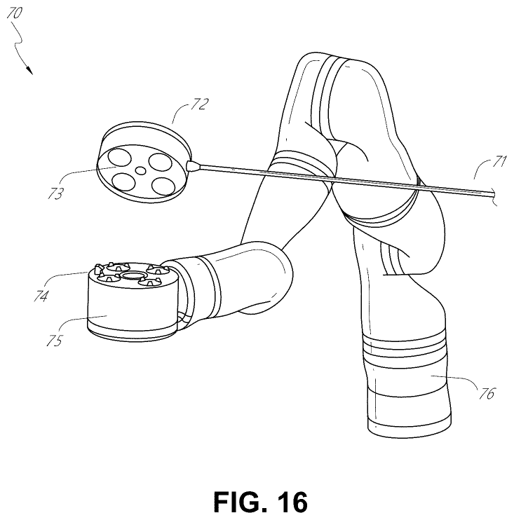

FIG. 16 illustrates an example medical instrument with a paired instrument driver. Like other instruments designed for use with a robotic system, medical instrument 70 comprises an elongated shaft 71 (or elongate body) and an instrument base 72. The instrument base 72, also referred to as an “instrument handle” due to its intended design for manual interaction by the physician, may generally comprise rotatable drive inputs 73, e.g., receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of instrument base 72 may share axes of rotation with the drive outputs 74 in the instrument driver 75 to allow the transfer of torque from drive outputs 74 to drive inputs 73. In some embodiments, the drive outputs 74 may comprise splines that are designed to mate with receptacles on the drive inputs 73.

The elongated shaft 71 is designed to be delivered through either an anatomical opening or lumen, e.g., as in endoscopy, or a minimally invasive incision, e.g., as in laparoscopy. The elongated shaft 71 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 74 of the instrument driver 75. When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons along the shaft 71. These individual tendons, such as pull wires, may be individually anchored to individual drive inputs 73 within the instrument handle 72. From the handle 72, the tendons are directed down one or more pull lumens along the elongated shaft 71 and anchored at the distal portion of the elongated shaft 71, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at distal end of the elongated shaft 71, where tension from the tendon cause the grasper to close.

In endoscopy, the tendons may be coupled to a bending or articulating section positioned along the elongated shaft 71 (e.g., at the distal end) via adhesive, control ring, or other mechanical fixation. When fixedly attached to the distal end of a bending section, torque exerted on drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing there between may be altered or engineered for specific purposes, wherein tighter spiraling exhibits lesser shaft compression under load forces, while lower amounts of spiraling results in greater shaft compression under load forces, but also exhibits limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft may comprise of a working channel for deploying surgical tools (or medical instruments), irrigation, and/or aspiration to the operative region at the distal end of the shaft 71. The shaft 71 may also accommodate wires and/or optical fibers to transfer signals to/from an optical assembly at the distal tip, which may include of an optical camera. The shaft 71 may also accommodate optical fibers to carry light from proximally located light sources, such as light emitting diodes, to the distal end of the shaft.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 16, the drive shaft axes, and thus the drive input axes, are orthogonal to the axis of the elongated shaft. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongated shaft 71. The resulting entanglement of such tendons may disrupt any control algorithms intended to predict movement of the flexible elongated shaft during an endoscopic procedure.

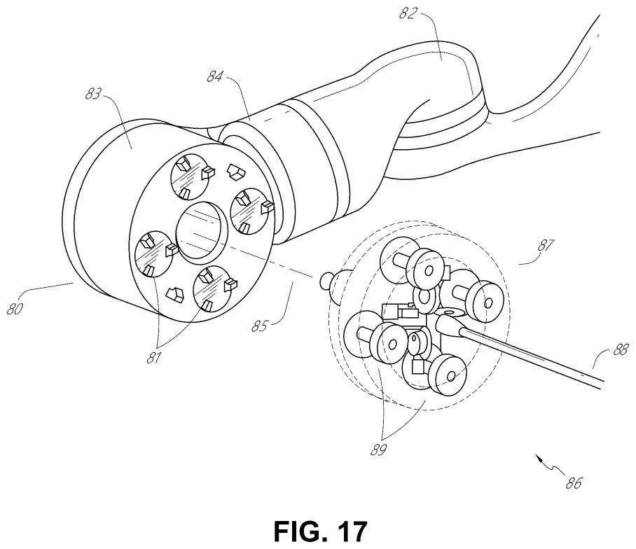

FIG. 17 illustrates an alternative design for an instrument driver and instrument where the axes of the drive units are parallel to the axis of the elongated shaft of the instrument. As shown, a circular instrument driver 80 comprises four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver. Power and controls signals may be communicated from the non-rotational portion 84 of the instrument driver 80 to the rotational assembly 83 through electrical contacts and may be maintained through rotation by a brushed slip ring connection (not shown). In other embodiments, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

Like earlier disclosed embodiments, an instrument 86 may comprise an elongated shaft portion 88 and an instrument base 87 (shown with a transparent external skin for discussion purposes) comprising a plurality of drive inputs 89 (such as receptacles, pulleys, and spools) that are configured to receive the drive outputs 81 in the instrument driver 80. Unlike prior disclosed embodiments, instrument shaft 88 extends from the center of instrument base 87 with an axis substantially parallel to the axes of the drive inputs 89, rather than orthogonal as in the design of FIG. 16.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

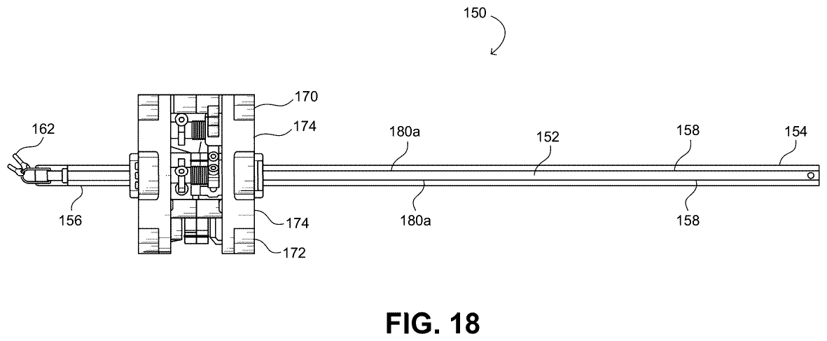

FIG. 18 illustrates an instrument having an instrument-based insertion architecture in accordance with some embodiments. The instrument 150 can be coupled to any of the instrument drivers discussed above. The instrument 150 comprises an elongated shaft 152, an end effector 162 connected to the shaft 152, and a handle 170 coupled to the shaft 152. The elongated shaft 152 comprises a tubular member having a proximal portion 154 and a distal portion 156. The elongated shaft 152 comprises one or more channels or grooves 158 along its outer surface. The grooves 158 are configured to receive one or more wires or cables 180 therethrough. One or more cables 180 thus run along an outer surface of the elongated shaft 152. In other embodiments, cables 180 can also run through the elongated shaft 152. Manipulation of the one or more cables 180 (e.g., via an instrument driver) results in actuation of the end effector 162.

The instrument handle 170, which may also be referred to as an instrument base, may generally comprise an attachment interface 172 having one or more mechanical inputs 174, e.g., receptacles, pulleys or spools, that are designed to be reciprocally mated with one or more torque couplers on an attachment surface of an instrument driver.

In some embodiments, the instrument 150 comprises a series of pulleys or cables that enable the elongated shaft 152 to translate relative to the handle 170. In other words, the instrument 150 itself comprises an instrument-based insertion architecture that accommodates insertion of the instrument, thereby minimizing the reliance on a robot arm to provide insertion of the instrument 150. In other embodiments, a robotic arm can be largely responsible for instrument insertion.

### E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

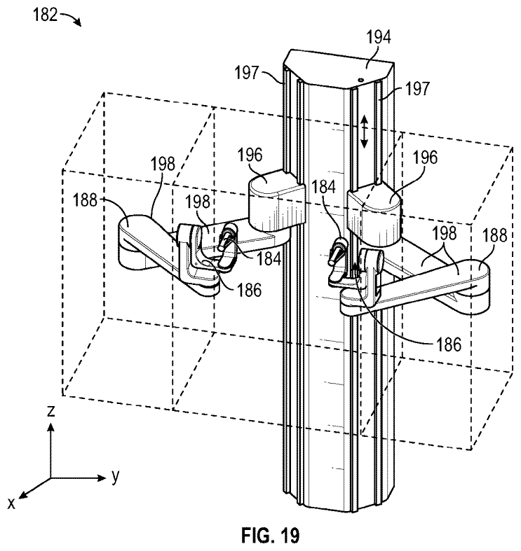

FIG. 19 is a perspective view of an embodiment of a controller 182. In the present embodiment, the controller 182 comprises a hybrid controller that can have both impedance and admittance control. In other embodiments, the controller 182 can utilize just impedance or passive control. In other embodiments, the controller 182 can utilize just admittance control. By being a hybrid controller, the controller 182 advantageously can have a lower perceived inertia while in use.

In the illustrated embodiment, the controller 182 is configured to allow manipulation of two medical instruments and includes two handles 184. Each of the handles 184 is connected to a gimbal 186. Each gimbal 186 is connected to a positioning platform 188.

As shown in FIG. 19, each positioning platform 188 includes a SCARA arm (selective compliance assembly robot arm) 198 coupled to a column 194 by a prismatic joint 196. The prismatic joints 196 are configured to translate along the column 194 (e.g., along rails 197) to allow each of the handles 184 to be translated in the z-direction, providing a first degree of freedom. The SCARA arm 198 is configured to allow motion of the handle 184 in an x-y plane, providing two additional degrees of freedom.

In some embodiments, one or more load cells are positioned in the controller. For example, in some embodiments, a load cell (not shown) is positioned in the body of each of the gimbals 186. By providing a load cell, portions of the controller 182 are capable of operating under admittance control, thereby advantageously reducing the perceived inertia of the controller while in use. In some embodiments, the positioning platform 188 is configured for admittance control, while the gimbal 186 is configured for impedance control. In other embodiments, the gimbal 186 is configured for admittance control, while the positioning platform 188 is configured for impedance control. Accordingly, for some embodiments, the translational or positional degrees of freedom of the positioning platform 188 can rely on admittance control, while the rotational degrees of freedom of the gimbal 186 rely on impedance control.

### F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as pre-operative mapping, computer vision, real-time EM tracking, and robot command data may be used individually or in combination to achieve a radiation-free operating environment. In other cases, where radiation-based imaging modalities are still used, the pre-operative mapping, computer vision, real-time EM tracking, and robot command data may be used individually or in combination to improve upon the information obtained solely through radiation-based imaging modalities.

FIG. 20 is a block diagram illustrating a localization system 90 that estimates a location of one or more elements of the robotic system, such as the location of the instrument, in accordance with an example embodiment. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1, the cart shown in FIGS. 1-4, the beds shown in FIGS. 5-14, etc.

As shown in FIG. 20, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Pre-operative mapping may be accomplished through the use of the collection of low dose CT scans. Pre-operative CT scans are reconstructed into three-dimensional images, which are visualized, e.g., as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 91 (also referred to as “preoperative model data” when generated using only preoperative CT scans). The use of center-line geometry is discussed in U.S. patent application Ser. No. 14/523,760, the contents of which are herein incorporated in its entirety. Network topological models may also be derived from the CT-images and are particularly appropriate for bronchoscopy.

In some embodiments, the instrument may be equipped with a camera to provide vision data 92. The localization module 95 may process the vision data to enable one or more vision-based location tracking. For example, the preoperative model data may be used in conjunction with the vision data 92 to enable computer vision-based tracking of the medical instrument (e.g., an endoscope or an instrument advance through a working channel of the endoscope). For example, using the preoperative model data 91, the robotic system may generate a library of expected endoscopic images from the model based on the expected path of travel of the endoscope, each image linked to a location within the model. Intra-operatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising of one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 93. The EM field generator (or transmitter) may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intra-operatively “registered” to the patient anatomy (e.g., the preoperative model) in order to determine the geometric transformation that aligns a single location in the coordinate system with a position in the pre-operative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during pre-operative calibration. Intra-operatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 20 shows, a number of other input data can be used by the localization module 95. For example, although not shown in FIG. 20, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94. Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Dynamic Flexible Scope Drive and Methods of Using the Same.

This application discloses robotic medical systems that include a robotic arm coupled to a flexible scope and a viewer for displaying a field of view of a surgical site derived from the flexible scope. The system can provide an indication of a position or an orientation of the flexible scope relative to a reference position.

In some embodiments, the robotic medical system can provide the indication via a user interface or display device of the robotic medical system. For example, in some embodiments, the robotic medical system provides a visual indication of a position or an orientation of the flexible scope (e.g., at a distal end of the scope).

In some embodiments, the robotic medical system is configured to operate the flexible scope in a particular mode of a plurality of modes (e.g., the robotic medical system may operate the flexible scope in a first mode of the plurality of modes at a first time and operate the flexible scope in a second mode, distinct from the first mode, of the plurality of modes at a second time that is distinct from the first time).

In some embodiments, the robotic medical system is configured to determine a position and/or orientation of the flexible scope. In some embodiments, the robotic medical system determines that a position or an orientation of the scope has changed and updates the indicator to indicate the changed position or orientation of the scope. In some embodiments, the robotic medical system updates the indicator in real time (e.g., while the scope is moving) to indicate the changed position or orientation of the scope.

### A. Robotic System.

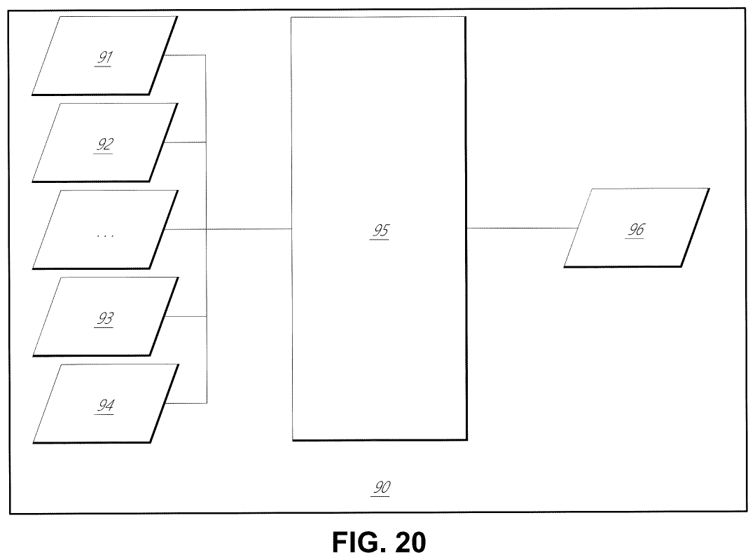

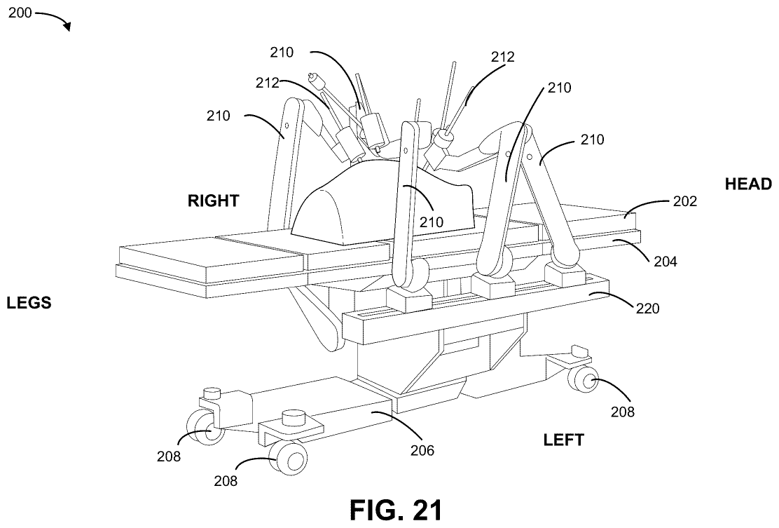

FIG. 21 illustrates an exemplary robotic medical system 200 according to some embodiments. In some embodiments, the robotic medical system 200 is a robotic surgery system. In the example of FIG. 21, the robotic medical system 200 comprises a patient support platform 202 (e.g., a patient platform, a table, a bed, etc.). The two ends along the length of the patient support platform 202 are respectively referred to as “head” and “leg”. The two sides of the patient support platform 202 are respectively referred to as “left” and “right.” The patient support platform 202 includes a support 204 (e.g., a rigid frame) for the patient support platform 202.

The robotic medical system 200 also comprises a base 206 for supporting the robotic medical system 200. The base 206 includes wheels 208 that allow the robotic medical system 200 to be easily movable or repositionable in a physical environment. In some embodiments, the wheels 208 are omitted from the robotic medical system 200 or are retractable, and the base 206 can rest directly on the ground or floor. In some embodiments, the wheels 208 are replaced with feet.

The robotic medical system 200 includes one or more robotic arms 210. The robotic arms 210 can be configured to perform robotic medical procedures as described above with reference to FIGS. 1-20. Although FIG. 21 shows five robotic arms 210, it should be appreciated that the robotic medical system 200 may include any number of robotic arms, including less than five or six or more.

The robotic medical system 200 also includes one or more bars 220 (e.g., adjustable arm support or an adjustable bar) that support the robotic arms 210. Each of the robotic arms 210 is supported on, and movably coupled to, a bar 220, by a respective base joint of the robotic arm. In some embodiments, and as described in FIG. 12, bar 220 can provide several degrees of freedom, including lift, lateral translation, tilt, etc. In some embodiments, each of the robotic arms 210 and/or the adjustable arm supports 220 is also referred to as a respective kinematic chain.

FIG. 21 shows three robotic arms 210 supported by the bar 220 that is in the field of view of the figure. The two remaining robotic arms are supported by another bar that is located across the other length of the patient support platform 202.

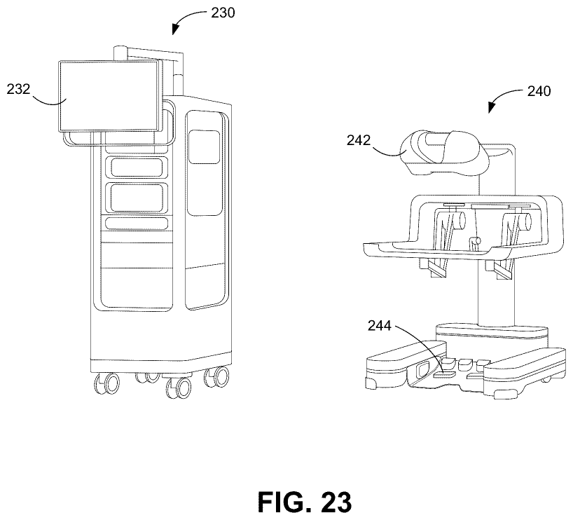

In some embodiments, the adjustable arm supports 220 can be configured to provide a base position for one or more of the robotic arms 210 for a robotic medical procedure. A robotic arm 210 can be positioned relative to the patient support platform 202 by translating the robotic arm 210 along a length of its underlying bar 220 and/or by adjusting a position and/or orientation of the robotic arm 210 via one or more joints and/or links (see, e.g., FIG. 23). In some embodiments, the bar pose can be changed via manual manipulation, teleoperation, and/or power assisted motion.

In some embodiments, the adjustable arm support 220 can be translated along a length of the patient support platform 202. In some embodiments, translation of the bar 220 along a length of the patient support platform 202 causes one or more of the robotic arms 210 supported by the bar 220 to be simultaneously translated with the bar or relative to the bar. In some embodiments, the bar 220 can be translated while keeping one or more of the robotic arms stationary with respect to the base 206 of the robotic medical system 200.

In the example of FIG. 21, the adjustable arm support 220 is located along a length of the patient support platform 202. In some embodiments, the adjustable arm support 220 may extend across a partial or full length of the patient support platform 202, and/or across a partial or full width of the patient support platform 202.

During a robotic medical procedure, one or more of the robotic arms 210 can also be configured to hold instruments 212 (e.g., robotically controlled medical instruments or tools, such as an endoscope and/or any other instruments (e.g., sensors, illumination instrument, cutting instrument, etc.) that may be used during surgery), and/or be coupled to one or more accessories, including one or more cannulas, in accordance with some embodiments.

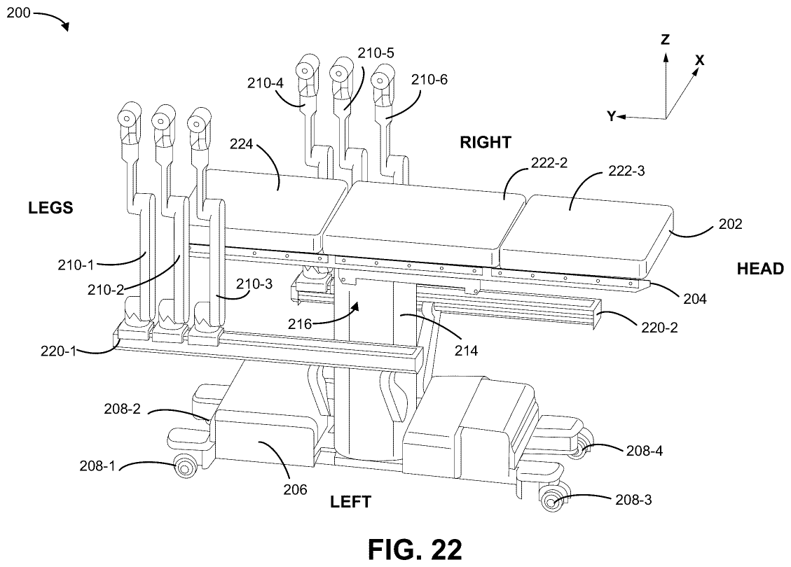

FIG. 22 illustrates another view of the exemplary robotic medical system 200 in FIG. 21 according to some embodiments. In this example, the robotic medical system 200 includes six robotic arms 210-1, 210-2, 210-3, 210-4, 210-5, and 210-6. The patient platform 202 is supported by a column 214 that extends between the base 206 and the patient platform 202. In some embodiments, the patient platform 202 comprises a tilt mechanism 216. The tilt mechanism 216 can be positioned between the column 214 and the patient platform 202 to allow the patient platform 202 to pivot, rotate, or tilt relative to the column 214. The tilt mechanism 216 can be configured to allow for lateral and/or longitudinal tilt of the patient platform 202. In some embodiments, the tilt mechanism 216 allows for simultaneous lateral and longitudinal tilt of the patient platform 202.

FIG. 22 shows the patient platform 202 in an untilted state or position. In some embodiments, the untilted state or position is a default position of the patient platform 202. In some embodiments, the default position of the patient platform 202 is a substantially horizontal position as shown in FIG. 22. As illustrated, in the untilted state, the patient platform 202 can be positioned horizontally or parallel to a surface that supports the robotic medical system 200 (e.g., the ground or floor). In some embodiments, the term “untilted” refers to a state in which the angle between the default position and the current position is less than a threshold angle (e.g., less than 5 degrees, or less than an angle that would cause the patient to shift on the patient platform, etc.). In some embodiments, the term “untilted” refers to a state in which the patient platform is substantially perpendicular to the direction of gravity, irrespective of the angle formed by the surface that supports the robotic medical system relative to gravity.

With continued reference to FIG. 22, in the illustrated example of the robotic medical system 200, the patient platform 202 includes a support 204. In some embodiments, the support 204 includes a rigid support structure or frame, and can support one or more surfaces, pads, or cushions 222. An upper surface of the patient platform 202 can include a support surface 224. During a medical procedure, a patient can be placed on the support surface 224.

FIG. 22 shows the robotic arms 210 and the adjustable arm supports 220 in an exemplary deployed configuration in which the robotic arms 210 reach above the patient platform 202. In some embodiments, due to the configuration of the robotic medical system 200, which enables stowage of different components beneath the patient platform 202, the robotic arms 210 and the arm supports 220 can occupy a space underneath the patient platform 202. Thus, in some embodiments, the tilt mechanism 216 has a low-profile and/or low volume in order to increase the space available for storage below.

FIG. 22 also illustrates an example, x, y, and z coordinate system that may be used to describe certain features of the embodiments disclosed herein. It will be appreciated that this coordinate system is provided for purposes of example and explanation only and that other coordinate systems may be used. In the illustrated example, the x-direction or x-axis extends in a lateral direction across the patient platform 202 when the patient platform 202 is in an untilted state. In some configurations, the x-direction extends across the patient platform 202 from one lateral side (e.g., the right side) to the other lateral side (e.g., the left side) when the patient platform 202 is in an untilted state. The y-direction or y-axis extends in a longitudinal direction along the patient platform 202 when the patient platform 202 is in an untilted state. That is, the y-direction extends along the patient platform 202 from one longitudinal end (e.g., the head end) to the other longitudinal end (e.g., the legs end) when the patient platform 202 is in an untilted state. In an untilted state, the patient platform 202 can lie in or be parallel to the x-y plane, which can be parallel to the floor or ground. In the illustrated example, the z-direction or z-axis extends along the column 214 in a vertical direction. In some embodiments, the tilt mechanism 216 is configured to laterally tilt the patient platform 202 by rotating the patient platform 202 about a lateral tilt axis that is parallel to the y-axis. The tilt mechanism 216 can further be configured to longitudinally tilt the patient platform 202 by rotating the patient platform 202 about a longitudinal tilt axis that is parallel to the x-axis.

In some embodiments, the robotic medical system 200 includes a tower 230 (e.g., tower viewer) or a physician console 240 (or both), as illustrated in FIG. 23. The tower 230 may correspond to the tower 30 described above, and may provide support for controls, electronics, fluidics, optics, sensors, and/or power for the patient support platform 202 and the physician console 240. In some embodiments, the tower 230 includes a display device 232. The display device 232 can include a user interface for displaying a surgical view obtained by one or more cameras 606 of the robotic medical system and/or one or more notifications to an operator of the robotic medical system 200. In some embodiments, the physician console 240 can include a display device 242 having a user interface used by the physician operator for operating the patient support platform 202. For example, the display device 242 may include a user interface for displaying a surgical view obtained by one or more cameras 606 of the robotic medical system and/or one or more notifications to an operator of the robotic medical system 200. The physician console 240 can provide both robotic controls and pre-operative and real-time information of a medical procedure to a physician operator. In some embodiments, the physician console 240 includes one or more input devices (e.g., buttons, switches, touch-sensitive surfaces, gimbals, etc.), such as a foot pedal 244.

### B. Robotic Arm.

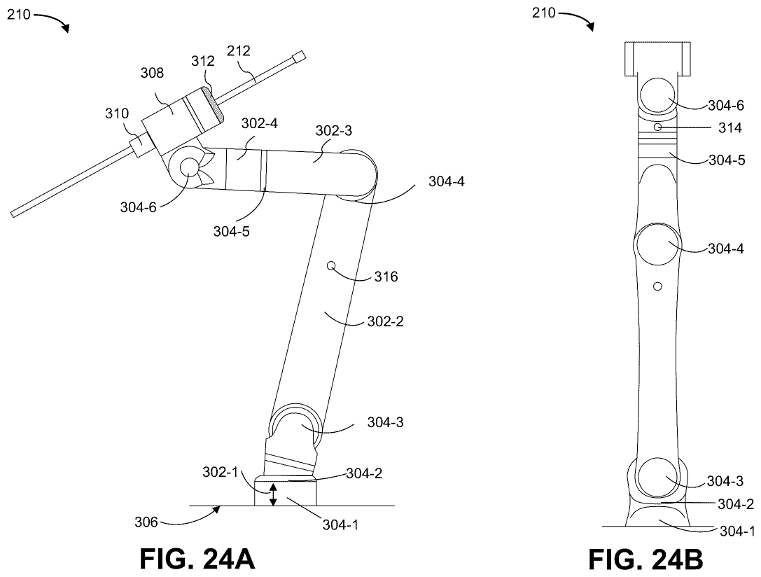

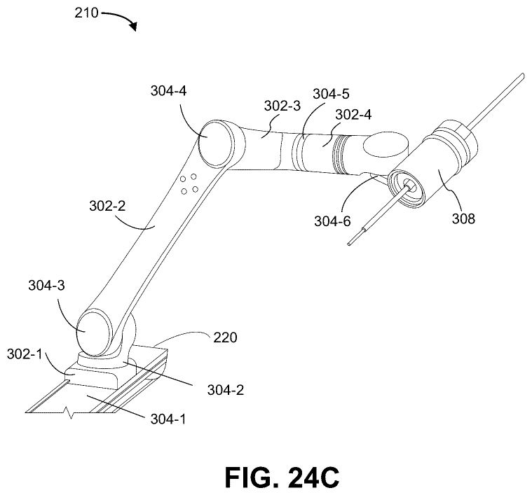

FIGS. 24A to 24C illustrate different views of an exemplary robotic arm 210 according to some embodiments.

FIG. 24A illustrates that the robotic arm 210 includes a plurality of links 302 (e.g., linkages). The links 302 (e.g., 302-1 through 302-4) are connected by one or more joints 304 (e.g., 304-1 through 304-5). Each of the joints 304 includes one or more degrees of freedom (DoFs).

In FIG. 24A, the joints 304 include a first joint 304-1 (e.g., a base joint or an A0 joint) that is located at or near a base 306 of the robotic arm 210. In some embodiments, the base joint 304-1 comprises a prismatic joint that allows the robotic arm 210 to translate along the bar 220 (e.g., along the y-axis). The joints 304 also include a second joint 304-2. In some embodiments, the second joint 304-2 rotates with respect to the base joint 304-1. The joints 304 also include a third joint 304-3 that is connected to one end of link 302-2. In some embodiments, the joint 304-3 includes multiple DoFs and facilitates both tilt and rotation of the link 302-2 tilt with respect to the joint 304-3.

FIG. 24A also shows a fourth joint 304-4 that is connected to the other end of the link 302-2. In some embodiments, the joint 304-4 comprises an elbow joint that connects the link 302-2 and the link 302-3. The joints 304 further comprise a pair of joints 304-5 (e.g., a wrist roll joint) and 304-6 (e.g., a wrist pitch joint), which is located on a distal portion of the robotic arm 210.

A proximal end of the robotic arm 210 may be connected to a base 306 and a distal end of the robotic arm 210 may be connected to an advanced device manipulator (ADM) 308 (e.g., a tool driver, an instrument driver, or a robotic end effector, etc.). The ADM 308 may be configured to control the positioning and manipulation of a medical instrument s (e.g., a tool, a scope, etc.).

The robotic arm 210 can also include a cannula sensor 310 for detecting presence or proximity of a cannula to the robotic arm 210. In some embodiments, the robotic arm 210 is placed in a docked state (e.g., docked position) when the cannula sensor 310 detects presence of a cannula (e.g., via one or more processors of the robotic medical system 200). In some embodiments, when the robotic arm 210 is in a docked position, the robotic arm 210 can execute null space motion to maintain a position and/or orientation of the cannula, as discussed in further detail below. Conversely, when no cannula is detected by the cannula sensor 310, the robotic arm 210 is placed in an undocked state (e.g., undocked position).

In some embodiments, and as illustrated in FIG. 24A, the robotic arm 210 includes an input or button 312 (e.g., a donut-shaped button, or other types of controls, etc.) that can be used to place the robotic arm 210 in an admittance mode (e.g., by depressing the button 312). The admittance mode is also referred to as an admittance scheme or admittance control. In the admittance mode, the robotic system 210 measures forces and/or torques (e.g., imparted on the robotic arm 210) and outputs corresponding velocities and/or positions. In some embodiments, the robotic arm 210 can be manually manipulated by a user (e.g., during a set-up procedure, or in between procedures, etc.) in the admittance mode. In some instances, by using admittance control, the operator need not overcome all of the inertia in the robotic medical system 200 to move the robotic arm 210. For example, under admittance control, when the operator imparts a force on the arm, the robotic medical system 200 can measure the force and assist the operator in moving the robotic arm 210 by driving one or more motors associated with the robotic arm 210, thereby resulting in desired velocities and/or positions of the robotic arm 210.

In some embodiments, the links 302 may be detachably coupled to the medical tool 212 (e.g., to facilitate ease of mounting and dismounting of the medical tool 212 from the robotic arm 210). The joints 304 provide the robotic arm 210 with a plurality of degrees of freedom (DoFs) that facilitate control of the medical tool 212 via the ADM 308. In an embodiment as shown in FIG. 22 including multiple robotic arms, each robotic arm can hold its own respective medical tool and pivot the medical tool about a remote center of motion.

FIG. 24B illustrates a front view of the robotic arm 210. FIG. 24C illustrates a perspective view of the robotic arm 210. In some embodiments, the robotic arm 210 includes a second input or button 314 (e.g., a push button) that is distinct from the button 312 in FIG. 24A, for placing the robotic arm 210 in an impedance mode (e.g., by a single press or continuous press and hold of the button 314). In this example, the button 314 is located between the joint 304-5 and the joint 304-6. The impedance mode is also referred to as impedance scheme or impedance control. In the impedance mode, the robotic medical system 200 measures displacements (e.g., changes in position and velocity) and outputs forces and/or torques to facilitate manual movement of the robotic arm. In some embodiments, the robotic arm 210 can be manually manipulated by a user (e.g., during a set-up procedure) in the impedance mode. In some embodiments, under the impedance mode, the operator's movement of one part of a robotic arm 210 may cause motion in one or more joints and/or links throughout the robotic arm 210.

In some embodiments, for admittance control, a force sensor or load cell can measure the force that the operator is applying to the robotic arm 210 and move the robotic arm 210 in a way that feels light. Admittance control may feel lighter than impedance control because, under admittance control, one can hide the perceived inertia of the robotic arm 210 because motors in the controller can help to accelerate the mass. In contrast, with impedance control, the user is responsible for most if not all mass acceleration, in accordance with some embodiments.

In some circumstances, depending on the position of the robotic arm 210 relative to the operator, it may be inconvenient to reach the button 312 and/or the button 314 to activate a manual manipulating mode (e.g., the admittance mode and/or the impedance mode). Accordingly, under these circumstances, it may be convenient for the operator to trigger the manual manipulation mode other than by buttons.

In some embodiments, the robotic arm 210 includes a single button (e.g., the button 312 or 314) that can be used to place the robotic arm 210 in the admittance mode and/or the impedance mode (e.g., by using different presses, such as a long press, a short press, press and hold etc.). In some embodiments, the robotic arm 210 can be placed in impedance mode by a user pushing on arm linkages (e.g., the links 302) and/or joints (e.g., the joints 304) and overcoming a force threshold. In some embodiments, the admittance mode and the impedance mode are common in that they both allow the user to grab the robotic arm 210 and command motion by directly interfacing with it.

In some embodiments, the robotic arm 210 includes an input control for activating an arm follow mode. For example, in some embodiments, the robotic arm 210 can include a designate touch point that is located on a link 302 or a joint 304 of the robotic arm (e.g., an outer shell of the link 302 or a button 316). User interaction (e.g., user touch, contact, etc.) with the designate touch point activates the arm follow mode. In some embodiments, the robotic arm 210 includes multiple touch points. User interaction with any (e.g., one or more) of the touch points activates the arm follow mode.

During a medical procedure, it can be desirable to have the ADM 308 of the robotic arm 210 and/or a remote center of motion (RCM) of the tool 212 coupled thereto kept in a static pose (e.g., position and/or orientation). An RCM may refer to a point in space where a cannula or other access port through which a medical tool 212 is inserted is constrained in motion. In some embodiments, the medical tool 212 includes an end effector that is inserted through an incision or natural orifice of a patient while maintaining the RCM. In some embodiments, the medical tool 212 includes an end effector that is in a retracted state during a setup process of the robotic medical system.

In some circumstances, the robotic medical system 200 can be configured to move one or more links 302 of the robotic arm 210 within a “null space” to avoid collisions with nearby objects (e.g., other robotic arms), while the ADM 308 of the robotic arm 210 and/or the RCM are maintained in their respective poses (e.g., positions and/or orientations). The null space can be viewed as the set of joint states through which a robotic arm 210 can move that does not result in movement of the ADM 308 and/or RCM, thereby maintaining the position and/or the orientation of the medical tool 212 (e.g., within a patient). In some embodiments, a robotic arm 210 can have multiple positions and/or configurations available for each pose of the ADM 308.

For a robotic arm 210 to move an instrument to a desired pose in space, in certain embodiments, the robotic arm 210 may have at least six DoFs-three DoFs for translation (e.g., X, Y, and Z positions) and three DoFs for rotation (e.g., yaw, pitch, and roll). In some embodiments, each joint 304 may provide the robotic arm 210 with a single DoF, and thus, the robotic arm 210 may have at least six joints to achieve freedom of motion to position the ADM 308 at any pose in space. To further maintain the ADM 308 of the robotic arm 210 and/or the remote center or motion in a desired pose, the robotic arm 210 may further have at least one additional “redundant joint.” Thus, in certain embodiments, the system may include a robotic arm 210 having at least seven joints 304, providing the robotic arm 210 with at least seven DoFs. In some embodiments, the robotic arm 210 may include a subset of joints 304 each having more than one degree of freedom thereby achieving the additional DoFs for null space motion. However, depending on the embodiment, the robotic arm 210 may have a greater or fewer number of DoFs.

Furthermore, as described with respect to FIG. 12, the bar 220 (e.g., adjustable arm support) can provide several degrees of freedom, including lift, lateral translation, tilt, etc. Thus, depending on the embodiment, a robotic medical system can have many more robotically controlled degrees of freedom beyond just those in the robotic arms 210 to provide for null space movement and collision avoidance. In a respective embodiment of these embodiments, the end effectors of one or more robotic arms (and any tools or instruments coupled thereto) and a remote center along the axis of the tool can advantageously maintain in pose and/or position within a patient.

A robotic arm 210 having at least one redundant DoF has at least one more DoF than the minimum number of DoFs for performing a given task. For example, a robotic arm 210 can have at least seven DoFs, where one of the joints 304 of the robotic arm 210 can be considered a redundant joint, in accordance with some embodiments. The one or more redundant joints can allow the robotic arm 210 to move in a null space to both maintain the pose of the ADM 308 and a position of an RCM and avoid collision(s) with other robotic arms or objects.

In some embodiments, the robotic medical system 200 can be configured to perform collision avoidance to avoid collision(s), e.g., between adjacent robotic arms 210, by taking advantage of the movement of one or more redundant joints in a null space. For example, when a robotic arm 210 collides with or approaches (e.g., within a defined distance of) another robotic arm 210, one or more processors of the robotic medical system 200 can be configured to detect the collision or impending collision (e.g., via kinematics). Accordingly, the robotic medical system 200 can control one or both of the robotic arms 210 to adjust their respective joints within the null space to avoid the collision or impending collision. In an embodiment including at least a pair of robotic arms, a base of one of the robotic arms and its end effector can stay in its pose, while links or joints therebetween move in a null space to avoid collisions with an adjacent robotic arm.

### C. Exemplary Surgical Settings.

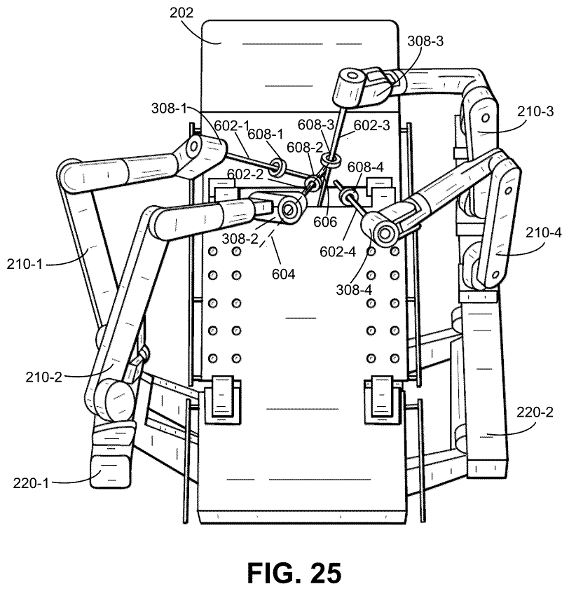

FIG. 25 illustrates a perspective view of a robotic medical system 200 that includes four robotic arms 210-1, 210-2, 210-3, and 210-4, in accordance with some embodiments. Each of the robotic arms 210-1, 210-2, 210-3, and 210-4 is coupled to a respective surgical tool 602 (e.g., 602-1 through 602-4, which may correspond to instrument 212) via a respective ADM 308 (e.g., tool driver), such as ADMs 308-1 through 308-4. A surgical tool 602 can be inserted into a patient via a respective port 608 located on the patient. As used herein, a port (e.g., a port location, a port of entry, an entry point, a port region, a port area, or a port position, etc.) refers to a position on a patient's body through which a medical tool/instrument (e.g., held by a robotic arm) can be inserted and constrained in motion. In some embodiments, the port corresponds to an incision point (or an incision region) that is made through the skin of the patient to facilitate a medical operation or procedure. In some embodiments, the port corresponds to a natural orifice, such as a mouth of the patient (e.g., for a bronchoscopy procedure). In some embodiments, the port corresponds to a medical device with an opening, placed at the incision point or the natural orifice to allow access to a surgical space through the opening. The view of the patient has been excluded from FIG. 25 in order to enhance the visibility of the robotic arms 210 and the surgical tools 602.

In FIG. 25, the robotic arm 210-2 is coupled to a camera 606. In this example, the camera 606 is coupled to the robotic arm 210—via a medical instrument 602-2 (e.g., an endoscope) (e.g., at a distal end of the medical instrument 602-2). In some embodiments, the camera 606 is a part of the medical instrument 602-2. In some embodiments, the camera 606 can be a standalone device (e.g., not part of a surgical instrument) that is coupled to a robotic arm (e.g., the camera 606 is distinct and separate from a medical instrument). In some embodiments, the camera 606 defines an axis 604 (e.g., an optical axis), which identifies an orientation of the camera 606. The camera 606 (or a scope) provides an image of a surgical site to facilitate control of surgical tools to perform a robotic medical procedure. For example, a robotically controllable endoscope of the robotic system can include a camera positioned at a distal tip thereof. The user can view an image from the camera of the endoscope in a viewer in order to facilitate control of the endoscope and/or other components of the robotic medical system. As another example, the robotic system may include one or more cameras laparoscopically or endoscopically inserted into a patient. The user can view images from the inserted cameras in order to facilitate control of one or more additional robotically controlled medical instruments, such as one or more additional laparoscopically inserted medical instruments.

In some embodiments, the robotic medical system 200 includes a coordinate system (e.g., a robot coordinate system, a coordinate frame, a system frame, etc. that may be a Cartesian or non-Cartesian coordinate system), and respective positions of the patient support platform 202, the robotic arms 210, the adjustable arm supports 220, and/or instruments 212 are represented as coordinates (e.g., x-, y-, and z-coordinates) on the coordinate system. For example, the robotic medical system 200 (e.g., one or more processors 380 of the robotic medical system 200) may be configured to identify positions and orientations of the patient support platform 202, the robotic arms 210, the adjustable arm supports 220, and/or instruments 212 based on coordinates in the coordinate system.

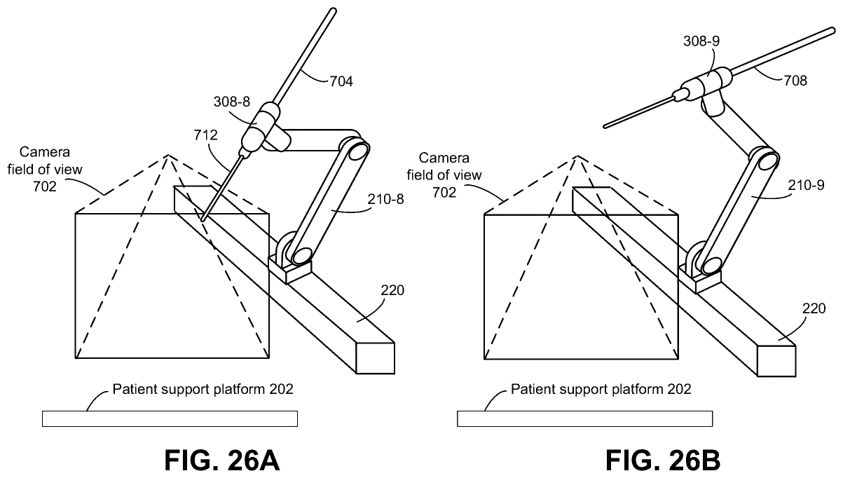

FIGS. 26A and 26B illustrate a camera field of view in a three-dimensional space in accordance with some embodiments. In some embodiments, the robotic medical system 200 determines the field of view based on a focal length of the camera 606 (e.g., the field of view may correspond to a space within a certain distance from a center of the field of view of the camera 606). In some embodiments, the robotic medical system 200 determines the field of view using data obtained by one or more sensors 388 of the robotic medical system 200, such as position (and orientation) data corresponding to the tip of an endoscope (e.g., a medical instrument) to which the camera 606 is coupled or position data corresponding to the camera 606 (e.g., the safety region may be determined to be located at a particular distance from the camera 606). In some embodiments, the robotic medical system 200 stores (e.g., within memory) information identifying the field of view of the camera 606 and utilizes the stored information to determine the field of view of the camera 606 based on the position and orientation of the camera 606. For example, the robotic medical system 200 may store information identifying a predefined height (e.g., several centimeters) and predefined base angles, slant angles, and/or edge angles of a three-dimensional volume, corresponding to the field of view, having a shape of a truncated rectangular pyramid. In another example, the robotic medical system 200 may store information identifying a predefined radius or diameter and a predefined distance (e.g., several centimeters) for a spherical volume, corresponding to the field of view, located at the predefined distance from the camera 606 and having the predefined radius or diameter. In some embodiments, the robotic medical system 200 determines the field of view according to image(s) obtained using the camera 606.

In FIGS. 26A and 26B, the camera field of view 702 (e.g., as seen by a camera that is coupled to another robotic arm) is represented by a three-dimensional space (e.g., a pyramid). It would be appreciated by one of ordinary skill in the art that the three-dimensional space of a camera field of view can be represented by other shapes such as a cone, a cylinder, a sphere, a tetrahedron, etc. In the example of FIG. 26A, the robotic arm 210-8 is coupled to a surgical tool 704 (e.g., a medical instrument) via ADM 308-8. The surgical tool 704 intersects (e.g., overlaps, is within, etc.) the three-dimensional space of the camera field of view 702. In contrast, FIG. 26B shows a robotic arm 210-9 holding a surgical tool 708 (e.g., via ADM 308-9) that does not intersect the three-dimensional space of the camera field of view 702 (e.g., because of the position and/or orientation of the tool 708).

### D. Flexible Scope

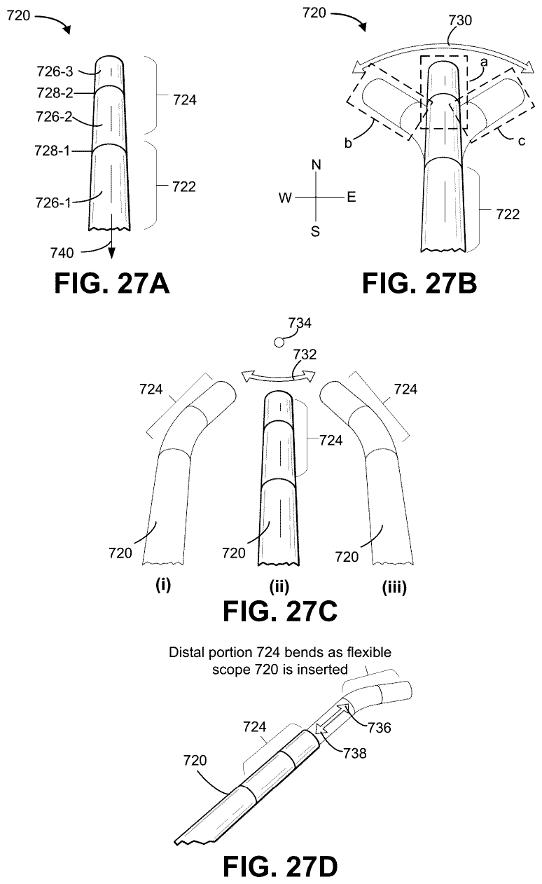

FIGS. 27A to 27D illustrate different views of a flexible scope 720 (e.g., a flexible laparoscope, a flexible endoscope etc.) in accordance with some embodiments. In some embodiments, the flexible scope is coupled to a robotic arm 210 of the robotic medical system 200.

FIG. 27A illustrates that the flexible scope 720 includes a distal end 724 (e.g., a distal portion) and an intermediate portion 722 (e.g., a portion positioned toward a proximal end 740 from the distal end 724) and. In some embodiments, the flexible scope 720 is coupled to a camera at the distal end 724. In some embodiments, the flexible scope 720 includes a plurality of links 726 (e.g., linkages). In some embodiments, the plurality of links 726 is connected by one or more joints 728. For example, in FIG. 27A, the flexible scope 720 includes links 726-1 through 726-3, which are connected by joints 728-1 and 728-2. In some embodiments, the plurality of links 726 includes four or more links.

In some embodiments, the flexible scope 720 comprises a rigid (e.g., not flexible) portion and a flexible portion. The rigid portion has a fixed (e.g., not flexible, cannot be varied) shape. The flexible portion has a shape that can be manipulated (e.g., varied, changed, adjusted, adapted, etc.).

In some embodiments, the rigid portion is located at a proximal end or toward the proximal end (e.g., the intermediate portion 722) of the flexible scope and the flexible portion is located at a distal end (e.g., the distal end 724) of the flexible scope. In some embodiments, the rigid portion is located at a distal end (e.g., the distal end 724) of the flexible scope and the flexible portion is located at a proximal end or toward the proximal end (e.g., the intermediate portion 722) of the flexible scope. In some embodiments, the flexible scope 720 includes two or more rigid portions (e.g., the links 726-1 and 726-3) coupled by a flexible portion (e.g., the link 726-2). In some embodiments, the rigid portion and the flexible portion are connected by a link (e.g., link 726-2) and/or a joint (e.g., joint 728-1 or 728-2), and the flexible portion can be moved (e.g., tilted, rotated, displaced, etc.) with respect to the rigid portion.

In some embodiments, the flexible scope 720 introduces additional degrees of freedom compared to a rigid scope. For example, in addition to the insertion, removal, pan, and rotate operations that are available on a rigid scope, at least a portion (e.g., a distal portion or an intermediate portion) of the flexible scope 720 is capable of being articulated (e.g., tilted, rotated, pivoted, flexed, caused to perform a rotary movement, etc.) relative to another portion of the flexible scope 720, thereby enabling a user to view a target anatomy at perspectives that are not achievable with a rigid scope. In some embodiments, the flexible scope 720 has a smaller volume than a rigid scope, thus freeing up space in a surgeon's workspace.

In some embodiments, the robotic system 200 (e.g., via processors 380) is configured to operate the flexible scope 720 in a plurality of modes (e.g., drive modes), such as an articulation mode (e.g., articulation drive mode), an orbit mode (e.g., orbit drive mode), and an automated insertion and retraction mode (e.g., automated insertion and retraction drive mode), as describe in further detail below. For example, in some embodiments, the robotic system 200 operates the flexible scope 720 in the articulation mode at a first time, in the orbit mode at a second time that is distinct from the first time, and in the automated insertion and retraction mode at a third time that is distinct from the first time and the second time. In some embodiments, the plurality of modes includes two of the aforementioned modes. In some embodiments, the plurality of modes includes four or more modes.

FIG. 27B illustrates operation of a flexible scope 720 in the articulation drive mode in accordance with some embodiments. In some embodiments, in the articulation drive mode, the distal portion 724 of the flexible scope 720 can be caused (e.g., by processors 380) to move (730) (e.g., articulate, perform an articulation movement, tilt, rotate, pivot, perform a rotatory movement, etc.) in an upward, downward, leftward, or rightward direction. For example, processors 380 may provide electrical signals (e.g., to one or more actuators, such as motors, or an actuation controller) to cause movement of the distal portion 724 of the flexible scope 720. FIG. 27B illustrates three poses or states of the flexible scope 720 as the distal portion 724 is caused to move or rotate (730) from an initial position or orientation (“a”) (e.g., aligned with the intermediate portion 722) (e.g., facing north), to a second position or a second orientation (“b”) (e.g., facing northwest), or to a third position or a third orientation (“c”) (e.g., facing northeast). In some embodiments, as illustrated in FIG. 27B, the intermediate portion 722 of the flexible scope 720 remains stationary in the articulation drive mode.

FIG. 27C illustrates operation of a flexible scope 720 in the orbit drive mode in accordance with some embodiments. In some embodiments, in the orbit drive mode, the flexible scope 720 (e.g., at the distal portion 724) can be caused (e.g., by processors 380) to move (732) (e.g., orbit) about a target point 734 (e.g., an orbit point) in space. For example, while the flexible scope 720 is operated in the orbit drive mode, as the flexible scope is moved (e.g., laterally), the distal end of the flexible scope 720 is rotated or tilted to face toward the target point 734. In FIG. 27C, three exemplary positions or poses of the flexible scope 720 are shown in (i), (ii), and (iii) as the distal end 724 of the flexible scope 720 orbits about the target point 734.

In some embodiments, the target point 734 is a fixed point in space. In some embodiments, the target point 734 is predefined (e.g., selected) by a user. In some embodiments, the target point 734 is predefined by the robotic system. In some embodiments, the target point 734 is automatically selected by the robotic medical system 200 (e.g., independently of a user input). For example, the target point 734 may be selected as a location of a particular organ or an anatomical structure of a patient.

FIG. 27D illustrates operation of a flexible scope 720 in the automated insertion and retraction drive mode in accordance with some embodiments. In some embodiments, in the automated insertion and retraction mode, the flexible scope 720 can be caused (e.g., by processors 380) to automatically (e.g., without manual force applied on the flexible scope 720) insert (e.g., move in direction 736) from a current position into the surgical site. In some embodiments, as illustrated in FIG. 27D, the distal portion 724 of the flexible scope bends as the flexible scope is inserted (e.g., along a sheath). In the automated insertion and retraction mode, the flexible scope 720 can also be caused (e.g., by processors 380) to automatically (e.g., without manual force applied on the flexible scope 720) retract (e.g., move in direction 738) from the surgical site to a port or to a cannula of the robotic arm to which the flexible scope 720 is coupled. In some embodiments, the distal portion 724 of the flexible scope 720 straightens as it is retracted.

A flexible scope is beneficial because it enables a user (e.g., a surgeon) to obtain different views of target anatomy that may not be possible with a rigid scope. However, operating (e.g., driving) a flexible scope in the various drive modes can be challenging. For example, in the articulation drive mode, it can be difficult for the user to identify a current shape or configuration of the flexible scope, or the degree or direction of articulation, or which direction the scope should be moved to return from a bent state to a straight state. In the orbit drive mode, it can be difficult for the user to determine a distance between the flexible scope and the target point about which the flexible scope is orbiting, or the position of the flexible scope in the orbit, or the shape of the flexible scope. In the automated insertion and retraction drive mode, it can be difficult for the user to understand a current state or position (along an insertion path) of the flexible scope as the flexible scope is being inserted or retracted, or whether the flexible scope is in a bent state or has been straightened. Accordingly, there is a need for a robotic medical system that can facilitate the user to visualize how a flexible scope is positioned or oriented.

### E. Exemplary User Interfaces.

In accordance with some embodiments, a robotic system includes a user interface that may be configured to display dynamic visual representations for the different drive modes of a flexible scope described above. In some embodiments, the user interface is configured to display dynamic visual representations for a flexible scope in accordance with a determination that a robotic arm is coupled to the flexible scope (e.g., regardless of drive modes).

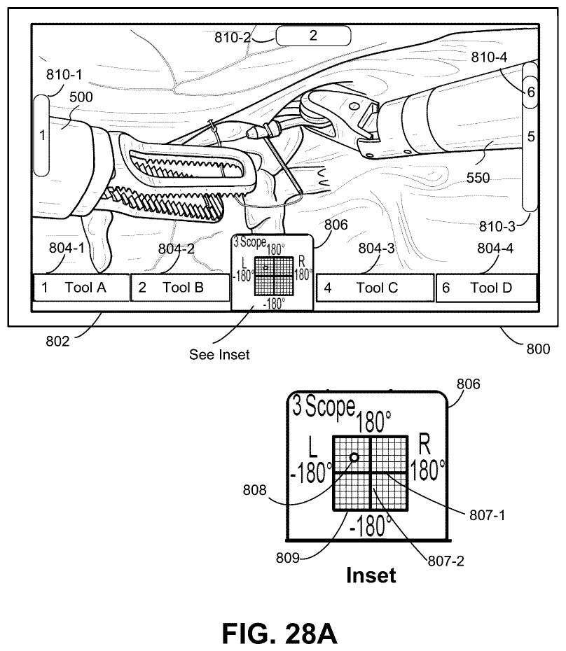

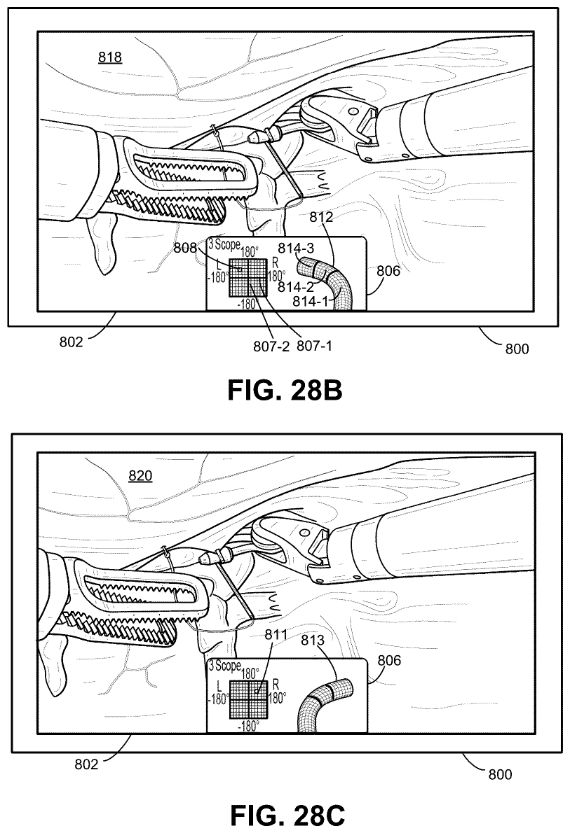

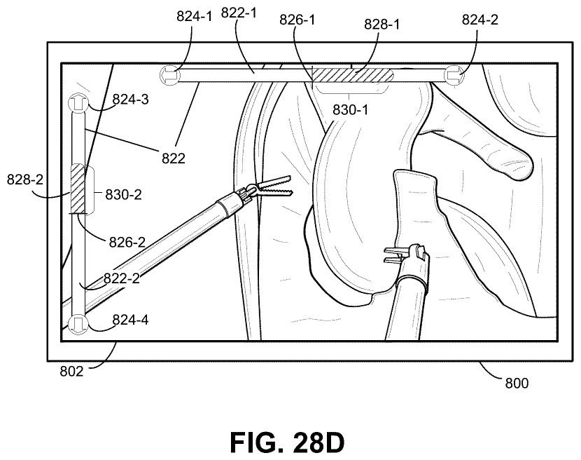

FIGS. 28A to 28D illustrate exemplary user interfaces displayed on a display device when the flexible scope is operating in an articulation drive mode, in accordance with some embodiments.

FIG. 28A shows a user interface 802 displayed on a display device 800 in accordance with some embodiments. In some embodiments, as shown in FIG. 28A, the user interface 802 includes an image or representation of a field of view of a camera (e.g., a view of a surgical site, such as a treatment site) or a scope (e.g., flexible scope 720). FIG. 28A also shows that the user interface 802 includes images or representations of medical instruments 500 and 550 located within the field of view of the camera or scope.

In some embodiments, as illustrated in FIG. 28A, the user interface 802 includes indicators 804 (e.g., visual indicators) that identify medical instruments (e.g., “Tool A,” “Tool B,” “Tool C,” “Tool D”) at the surgical site. In some embodiments, the indicators 804 include a number associated with a robotic arm coupled with the respective medical instrument. For example, the indicator 804-2 includes a number “2” indicating that Tool B is coupled to a second robotic arm of the robotic medical system.

In some embodiments, as illustrated in FIG. 28A, the user interface 802 includes an indicator 806 (e.g., a visual indicator, a scope tab, etc.) for indicating a position and/or orientation of the flexible scope (e.g., flexible scope 720), or a portion thereof (e.g., a distal end 724 of the flexible scope 720) relative to a reference position. The reference position can be a neutral position (e.g., the position corresponding to the fully straightened scope, a position determined based on an insertion port, etc.). In some embodiments, the indicator 806 has an initial size that is similar to the indicators 804, and subsequently expands (e.g., to a size as illustrated in FIG. 28A) when (e.g., in accordance with a determination by the robotic medical system that) the camera coupled to the flexible scope is or has become active.

The inset in FIG. 28A shows that the indicator 806 includes graphical elements such as a grid 809 that includes a reference axis 807-1 (e.g., a horizontal axis), a reference axis 807-2 (e.g., a vertical axis), and a marker 808 (e.g., a dot, a point, etc.). The marker 808 indicates a direction and/or extent of the articulation (e.g., movement, bending, pivoting, etc.) of the flexible scope. In the example of FIG. 28A, the indicator 806 indicates that a distal end of the flexible scope is articulated (e.g., moved, pivoted, bent, etc.) upwards and leftwards relative to its reference position by showing the marker 808 positioned above and to the left of an origin formed by the reference axes 807-1 and 807-2 (e.g., an intersection of the reference axes 807-1 and 807-2).

In some embodiments, the indicator 806 includes a number associated with a robotic arm coupled with the flexible scope. For example, FIG. 28A shows that the indicator 806 includes the number “3”, which indicates that that the flexible scope is coupled to a third robotic arm of the robotic medical system.

In some embodiments, as illustrated in FIG. 28A, the user interface 802 includes indicators 810 (e.g., along a periphery of the user interface 802 or a portion thereof) for indicating a position (or direction) of a medical instrument relative to the field of view. For example, in FIG. 28A, the indicator 810-1 is located along a left edge of the user interface 802, indicating that the medical instrument 500 is positioned on a left side of the field of view. In some embodiments, as shown in FIG. 28A, the indicator 810-1 includes information identifying the medical instrument 500 (e.g., a number associated with a robotic arm coupled with the medical instrument 550, such as the number “1” indicating a first robotic arm).

In some embodiments, the indicator 810 (or any other indicator for any medical tool) is displayed when the corresponding medical tool is located outside the field of view. In some embodiments, the indicator 810 (or any other indicator for any medical tool) is displayed regardless of whether the corresponding medical tool is located within or outside the field of view.

FIG. 28B shows that in some embodiments, the indicator 806 includes a representation 812 (e.g., a real-time representation or a three-dimensional representation or rendering) corresponding to a shape of the flexible scope, to further assist a user in understanding the shape of the scope. In some embodiments, the representation 812 includes renderings of links 814. In some embodiments, the number of links 814 (e.g., 814-1 through 814-3) in the representation 812 correspond to the number of links (e.g., links 726-1 through 726-3) of the flexible scope.

The transition between FIG. 28B and FIG. 28C shows a change in the articulation of the distal end of the flexible scope, from an upward and leftward position in FIG. 28B to an upward and rightward position in FIG. 28C. In some embodiments, in response to detecting a change in the articulation of the flexible scope, the user interface 802 (e.g., via processors 380) updates one or more graphical elements of the indicator 806 to reflect the changed position and/or shape of the scope. For example, in FIG. 28C, the user interface 802 displays an updated graphical element (e.g., marker) 811 that is positioned above and to the right of the origin formed by the reference axes 807-1 and 807-2, to indicate that the distal end of the scope has moved (articulated) to an upward and rightward position. FIG. 28C also shows the user interface 802 display with an updated representation 813 that reflects the current shape of the flexible scope. As illustrated in FIGS. 28B and 28C, the indicator 806 may operate or function as a dynamic indicator that changes in real time as the scope moves.

In some embodiments, the user interface 802 displays a representation of a field of view of the camera of the flexible scope that is updated in response to movement of the flexible scope. For example, in FIG. 28C, when the flexible scope is articulated up and to the right relative to its reference position, the user interface 802 displays an updated representation 820 of a field of view of the camera that has been translated leftward (in connection with the rightward movement of the distal end of the flexible scope) compared to the representation 818 of the field of view of the camera in FIG. 28B.

FIG. 28D illustrates a user interface 802 that includes indicator(s) 822 (e.g., bars, articulation bars, etc.), in accordance with some embodiments. In this example, the indicator(s) 822 include a horizontal bar 822-1 and a vertical bar 822-2. In some embodiments, the horizontal bar 822-1 includes a graphical element (e.g., an arrow) 824-1 representing a leftward articulation direction relative to a reference position 826-1 (e.g., center of the horizontal bar) and a graphical element (e.g., an arrow) 824-2 representing a rightward articulation direction relative to the reference position 826-1. In some embodiments, the vertical bar 822-2 includes a graphical element (e.g., an arrow) 824-3 representing an upward articulation direction relative to a reference position 826-2 (e.g., the center of the vertical bar) and a graphical element (e.g., arrow) 824-4 representing a downward articulation direction relative to the reference position 826-2.

In some embodiments, the indicator(s) 822 include a graphical element 828-1 representing a direction of the movement of the distal end of the flexible scope along the horizontal direction. In some embodiments, the indicator 822 includes a graphical element 828-2 representing a direction of the movement of the distal end of the flexible scope along the vertical direction. In the example of FIG. 28D, the distal end of the flexible scope is in a rightward and upward direction relative to its reference position. Accordingly, in the user interface 802 of FIG. 28D, the graphical element 828-1 is positioned to the right of the reference position 826-1 and the graphical element 828-2 is positioned above the reference position 826-2. In some embodiments, the distal end of the flexible scope moves (e.g., articulates, bends, flexes, etc.) in a rightward and upward direction relative to its reference position, and the user interface 802 indicates the direction of the movement with the graphical element 828-1 and the graphical element 828-2.

In some embodiments, each of the graphical elements 828-1 and 828-2 has (or is characterized by) a respective length representing an extent of the articulation of the flexible scope. In some embodiments, the lengths of the graphical elements 828-1 and 828-2 is updated dynamically (e.g., in real time) as the scope is moved. For example, the graphical element 828-1 has a length 830-1 that increases (e.g., in real time) as the scope is articulated rightwards, away from the reference position, and decreases as the scope is articulated leftwards toward the reference position. The element 828-2 has a length 830-2 that increases (e.g., in real time) as the scope is articulated upwards, away from the reference position, and decreases as the scope is articulated downwards toward the reference position.

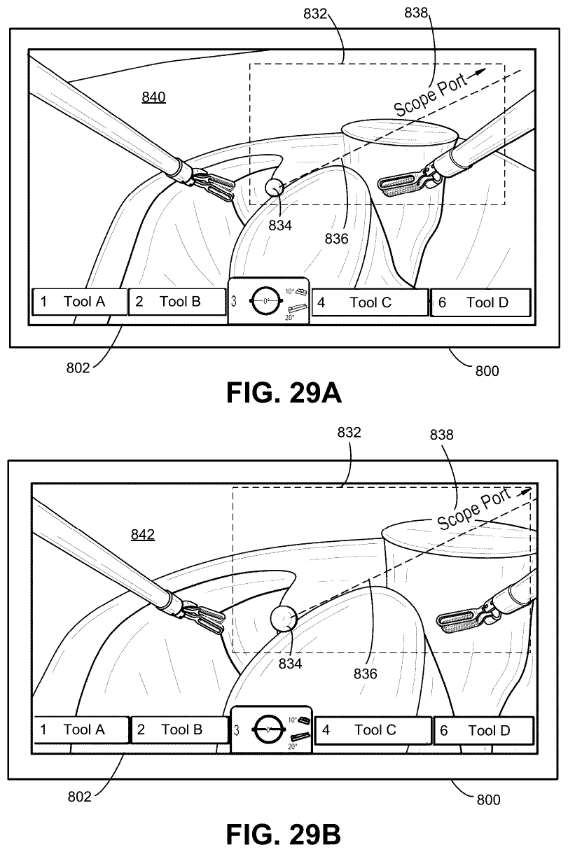

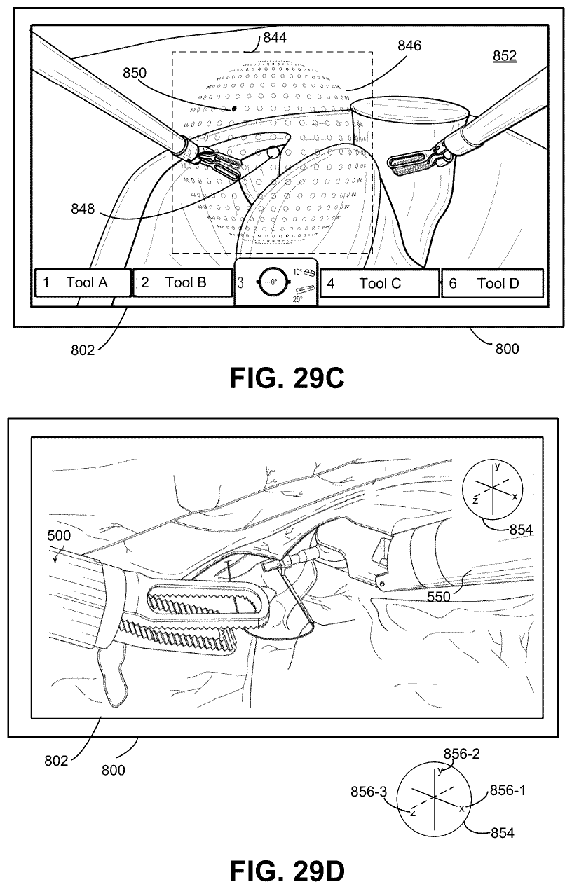

FIGS. 29A to 29D illustrate exemplary user interfaces displayed on a display device when the flexible scope is operating in an orbit drive mode, in accordance with some embodiments.

FIGS. 29A and 29B show a user interface 802 that includes an indicator 832. In some embodiments, the indicator 832 includes a graphical element 834 (e.g., a point, a sphere, a cross, an “X” symbol, an open circle, a closed circle, etc.) representing an orbit point (e.g., a target point, such as target point 734, etc.) about which the flexible scope is orbiting.

During surgery, a surgeon may drive an input device on the surgeon console 240 to control a depth of insertion of the flexible scope into the surgical site. In some embodiments, the graphical element 834 has (e.g., is characterized by) a dimension (e.g., a length, a width, a height, a diameter, etc.) that may be updated (e.g., varied) according to the change in the depth of insertion of the flexible scope. For example, the transition from FIG. 29A to FIG. 29B illustrates the flexible scope moving closer to the surgical site. In some embodiments, as the flexible scope moves closer to the surgical site (or moves toward a target organ, such as an organ that is positioned at the center of the field of view of the camera), the graphical element 834 that is displayed in the user interface 802 increases in diameter (e.g., is enlarged), as illustrated in the transition from FIG. 29A to FIG. 29B. In some embodiments, a smaller diameter of the graphical element 834 in FIG. 29A represents a larger distance between the scope and the surgical site, whereas a larger diameter of the graphical element 834 in FIG. 29B represents a smaller distance between the scope and the surgical site.

In some embodiments, the user interface 802 displays a representation of a field of view of the camera that varies according to a depth of insertion of the flexible scope 720 into the surgical site. For example, in FIG. 29B, the user interface 802 displays a representation 842 of the field of view of the camera that has a higher magnification compared to a magnification of the representation 840 of the field of view as depicted in FIG. 29A.

In some embodiments, as illustrated in FIGS. 29A and 29B, the indicator 832 includes a graphical element 836 (e.g., a line, a dashed line, etc.) representing a line of sight from the orbit point (or the target point) to a port corresponding to the flexible scope (e.g., the port through which the flexible scope is inserted). In some embodiments, the indicator 832 includes a graphical element 838 for indicating a direction to the scope port. In some embodiments, when the scope port, the camera, and the orbit point align (e.g., the port is behind the camera and the orbit point is in front of the camera along a common line of sight), the graphical element 836 is omitted. For example, the graphical element 836 (e.g., along with the graphical element 838) may converge or merge with the graphical element 834 so that the graphical element 836 is no longer displayed in the user interface 802 when the scope port, the camera, and the orbit point align.

FIG. 29C illustrates the user interface 802 with an orbit sphere in accordance with some embodiments. In this example, the user interface 802 displays an indicator 844 that includes a graphical element 846 (e.g., a sphere, a 3D sphere, a globe, etc.) representing an orbit sphere that depicts a range (e.g., a full range) of orbit motion of the flexible scope. In some embodiments, the indicator 844 includes a graphical element 848 (e.g., a first circle, an open circle, etc.) that represents an orbit point about which the flexible scope is orbiting. In some embodiments, the indicator 844 includes a graphical element 850 (e.g., a marker, a second circle, a closed circle, etc.) that represents a position of the flexible scope (e.g., in real-time) in the orbit sphere. In the example of FIG. 29C, the distal end of the scope is at the top and left position relative to the orbit point. In some embodiments, when the scope is aligned with the orbit point (e.g., as depicted in FIG. 27C as configuration (ii)), the graphical element 850 ceases to be displayed. For example, the graphical element 850 may converge or merge with the graphical element 848 so that the graphical element 836 is no longer displayed in the user interface 802 when the scope port, the camera, and the orbit point align.

In some embodiments, the graphical element 846 has a size (e.g., diameter, length, dimension, etc.) that correlates with a distance between the scope and the target point. In some embodiments, the size of the graphical element 846 is dynamic and varies (e.g., in real time) according to the distance between the scope and the target point. For example, the size (e.g., diameter) of the graphical element 846 increases as the scope is inserted and moved toward the surgical site (e.g., when the distance between the scope and the target point decreases) and decreases as the scope is retracted away from the surgical site (e.g., when the distance between the scope and the target point increases). In some embodiments, in accordance with the change in the size of the graphical element 846, the user interface 802 also displays graphical elements 848 and 850 at updated positions on the graphical element 846. The overlay of the graphical elements 846, 848, and 850 over the surgical field of view provides surgeons with insight as to the position and configuration of the scope, and how the scope can be straightened (e.g., by adjusting the scope from a position corresponding to the representation 850 to a position corresponding to the representation 848).

FIG. 29D illustrates the user interface 802 with a 3D axis indicator 854 according to some embodiments. The indicator 854 includes graphical elements 856 representing axes (e.g., an x-axis 856-1, a y-axis 856-2, and a z-axis 856-3). The orientation or rotation of the axes 856 represents an orientation or a position of the flexible scope relative to the port. In some embodiments, the user interface displays the indicator 854 with the graphical elements 856 in accordance with a determination that the scope and the orbit point are not in alignment (e.g., as depicted in FIG. 27C as configurations or poses (i) and (iii)). In some embodiments, when the scope aligns with the orbit point (e.g., the scope is a straight scope), the graphical element 856-3 ceases to be displayed or becomes a point.

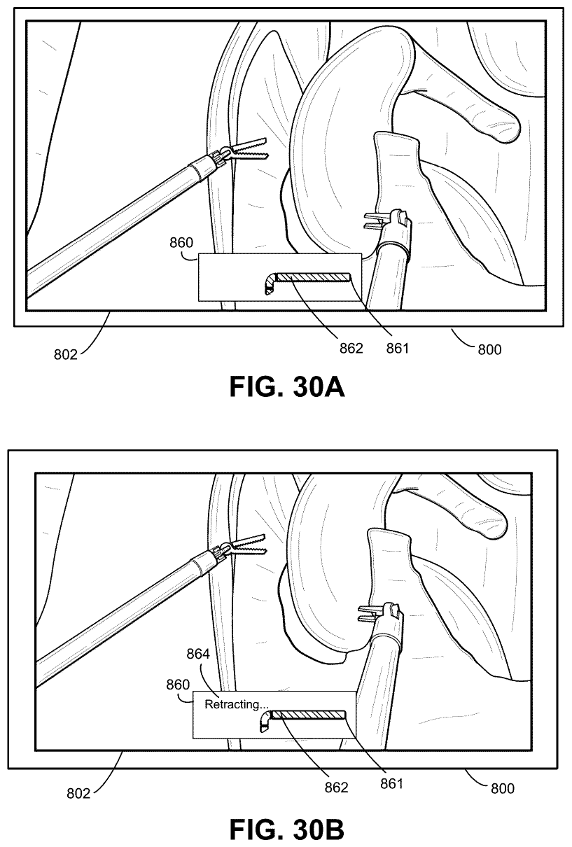

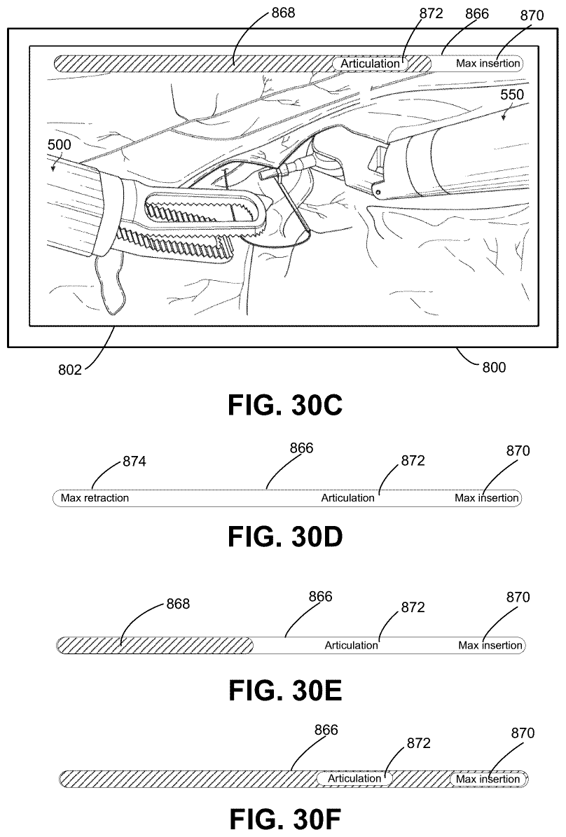

FIGS. 30A to 30C illustrate exemplary user interfaces displayed on a display device when the flexible scope is operating in an automated insertion and retraction mode, in accordance with some embodiments.

FIG. 30A illustrates display of an indicator 860 in the user interface 802. In some embodiments, the indicator 860 includes a graphical element 861 corresponding to a shape (e.g., an outline) of the flexible scope that is coupled to the robotic arm and used in the present surgery. In this example, the flexible scope is configured as a flexible scope that is articulated 90°. The indicator 860 also includes a graphical element 862 (e.g., shading, rendering, etc.) overlaid on the graphical element 861 and representing a real-time position or state of the scope. In the example of FIG. 30A, the scope is fully inserted and therefore the graphical element 862 completely fills (e.g., completely overlays) the graphical element 861.

FIG. 30B illustrates display of the indicator 860 in the user interface 802 as the scope is partially retracted. In FIG. 30B, the graphical element 862 partially fills the graphical element 861. The shaded portion of the scope (e.g., collectively represented by the graphical elements 861 and 862) represents the current shape and position of the scope in real time, and is dynamically updated (e.g., as the scope continues to retract, or is re-inserted).

In some embodiments, the user interface 802 displays a text element 864 such as “Inserting” or “Retracting” to indicate whether the scope is being inserted or retracted.

FIG. 30C illustrates a progress bar according to some embodiments. In this example, the user interface 802 displays an indicator 866 (e.g., a bar) that includes a graphical element 868 (e.g., a bar, a shaded portion, shading, rendering, etc.) indicating an extent of insertion and/or retraction of the scope. In some embodiments, the indicator 866 includes a graphical element 870 (e.g., a text element) corresponding to a fully inserted (e.g., maximum insertion) position of the scope. In some embodiments, the indicator 866 includes a graphical element 872 (e.g., an icon, a text element, etc.) that indicates whether the flexible scope is articulated. For example, in some implementations, the graphical element 872 is visually emphasized (e.g., lit up, highlighted, etc.) when or while the flexible scope is articulated. In the example of FIG. 30C, the flexible scope is in a partially inserted and articulated state. In this example, the graphical element 868 indicates the extent of the insertion and the visual emphasis of the graphical element 872 indicates that the scope is articulated.

FIGS. 30D-30F illustrate examples of the indicator 866 indicating different states of the scope.

FIG. 30D illustrates an indicator 866 corresponding to a fully retracted and straight state of the flexible scope in accordance with some embodiments. In some embodiments, the indicator 866 includes a graphical element 874 (e.g., a text element) corresponding to a fully retracted position of the scope. In some embodiments, the indicator 866 includes a graphical element 870 (e.g., a text element) corresponding to a fully inserted position of the scope, as described with respect to FIG. 30C. In FIG. 30D, the indicator 866 also includes the graphical element 872. In some embodiments, the graphical element 872 is visually deemphasized as shown in FIG. 30D or omitted (or ceases to be displayed) to indicate that the scope is not articulated.

FIG. 30E illustrates an indicator 866 corresponding to a partially inserted and straight (e.g., non-articulated) state of the flexible scope in accordance with some embodiments. In FIG. 30E, the graphical element 868 indicates the extent of the insertion (e.g., a partial insertion). Similar to the indicator 866 shown in FIG. 30D, the graphical element 872 may be visually deemphasized as shown in FIG. 30E or alternatively, omitted (or cease to be displayed) to indicate that the scope is not articulated.

FIG. 30F illustrates an indicator 866 corresponding to a fully inserted and articulated (e.g., partially articulated, fully articulated, etc.) state of the flexible scope in accordance with some embodiments. In FIG. 30F, the graphical element 870 indicates the extent of insertion of the scope. For example, as shown in FIG. 30F, the graphical element 870 is visually emphasized to indicate that the scope is fully inserted. In addition, the graphical element 872 indicates whether the scope is articulated. For example, as shown in FIG. 30F, the graphical element 872 is visually emphasized to indicate that the scope is articulated.

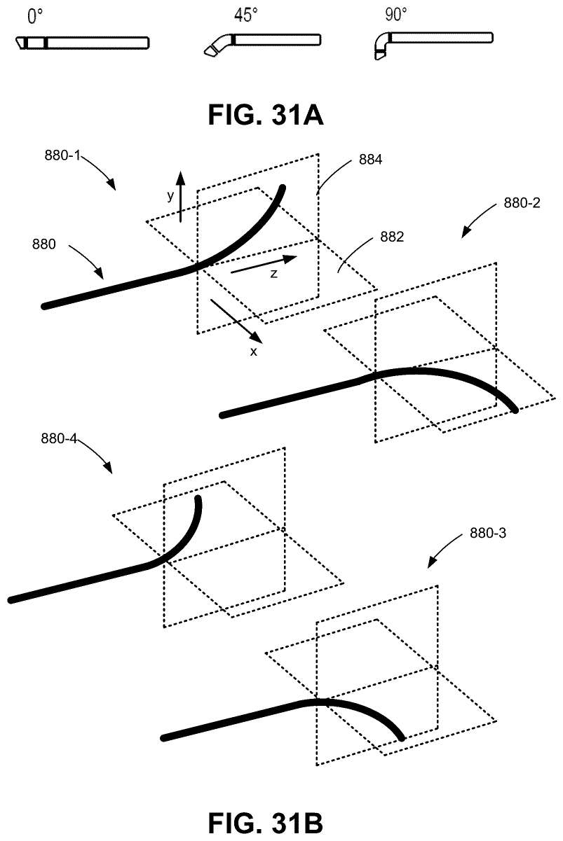

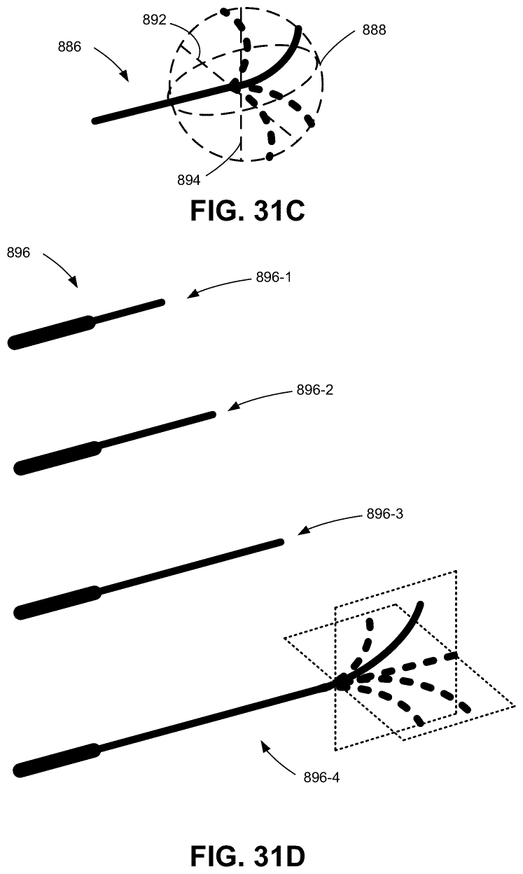

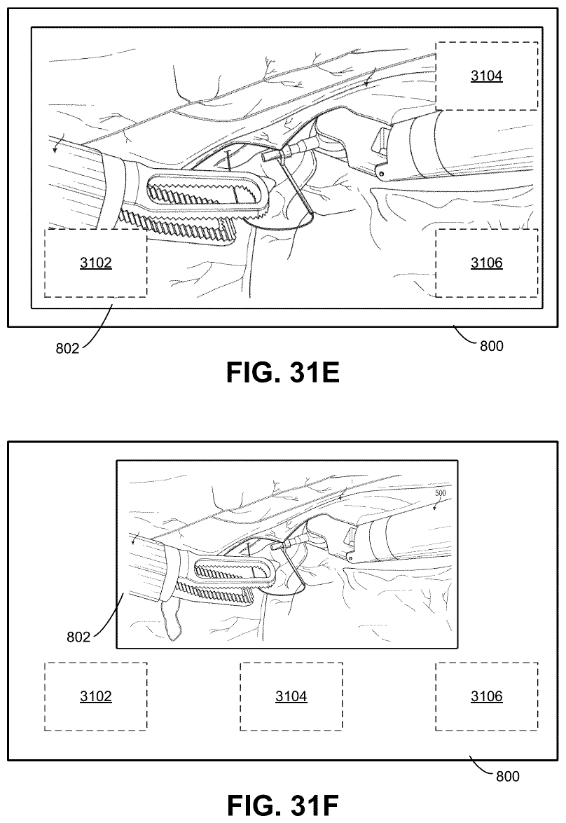

FIG. 31A illustrates different configurations of flexible scopes, such as a scope that is articulated 0°, 45°, and 90°, in accordance with some embodiments. In some embodiments, the shape or configuration of the scope at the time retraction or insertion is initiated (e.g., a 0° scope, a 45° scope, or a 90° scope) is displayed in the user interface 802 (e.g., as shown in FIGS. 30A-30B) while the progress of the insertion or retraction is indicated with the graphical element 862.

FIGS. 31B-31F illustrate exemplary user interface elements displayed on a display device to indicate a status of the flexible scope in accordance with some embodiments.

FIG. 31B illustrates an indicator 880 in accordance with some embodiments. In some embodiments, the indicator 880 is shown in a graphical projection (e.g., a non-primary graphical projection). In some embodiments, the indicator 880 is shown in a perspective view, an oblique view, or an auxiliary view, such as an isometric view (as shown in FIG. 31B), a diametric view, or a trimetric view. In some embodiments, the indicator 880 includes, or is displayed with, one or more reference planes (e.g., x-z plane 882 and y-z plane 884) or graphical representations thereof. In some embodiments, the one or more reference planes are semi-transparent (e.g., translucent). This facilitates concurrent display of the position of the scope relative to the one or more reference planes. FIG. 31B shows the indicator 880 in different states (e.g., 880-1 through 880-4). For example, the indicator 880 in state 880-1 represents a scope flexing upward (e.g., toward the direction of the y-axis), the indicator 880 in state 880-2 represents the scope flexing rightward (e.g., toward the direction of the x axis), the indicator 880 in state 880-3 represents the scope flexing downward (e.g., against the direction of the y axis), and the indicator 880 in state 880-4 represents the scope flexing leftward (e.g., against the direction of the x axis). In some embodiments, the indicator 880 is placed in different states representing flexing of the scope in a direction that is non-parallel and non-perpendicular to the reference planes.

FIG. 31C illustrates an indicator 886 in accordance with some embodiments. The indicator 886 shown in FIG. 31C is similar to the indicator 880 shown in FIG. 31B. However, in FIG. 31C, the indicator 886 is shown with a reference user interface element 888. In some embodiments, the reference user interface element 888 is depicted as a two-dimensional-like representation. In some embodiments, the reference user interface element 888 has a round shape (e.g., a circle, an ellipse, an oval, etc.). In some embodiments, the reference user interface element 888 is depicted as a three-dimensional-like representation. In some embodiments, the reference user interface element 888 is a graphical representation of a sphere, a dome, or a half-dome. In some embodiments, the reference user interface element 888 includes one or more axis indicators (e.g., x-axis indicator 892 and y-axis indicator 894). In some embodiments, the reference user interface element 888 (and one or more axis indicators) are semi-transparent (e.g., translucent). This facilitates concurrent display of the position of the scope relative to the reference user interface element 888 and one or more axis indicators. Although FIG. 31C shows representations of the indicator 886 in multiple states (e.g., the indicator 886 in a state representing a scope flexing upward is shown with a solid line, the indicator 886 in states representing the scope flexing leftward, rightward, and downward are shown with dashed lines), in some embodiments, the indicator 886 in only a single state is displayed (e.g., the indicator 886 represented by the solid line is shown without the dashed lines to represent the state of the scope flexing upward). In some embodiments, a portion of the reference user interface element 888 is visually distinguished (e.g., highlighted) to indicate an orientation or a pose of the scope. For example, a portion (e.g., one of four predefined quarters) of the reference user interface element 888 (e.g., a half-dome) is visually distinguished (e.g., in transparency, color, etc. from those of the rest of the four predefined quarters of the half-dome) to highlight an orientation or state of the flexible scope (or an orientation of state that the flexible scope is moving into).

FIG. 31D illustrates an indicator 896 in accordance with some embodiments. The indicator 896 shown in FIG. 31D is similar to the indicator 880 shown in FIG. 31B except that the indicator 896 shows insertion and/or progress of a scope in addition to the flexing of the scope. For example, the indicator 896 in states 896-1, 896-2, and 896-3 indicates different degrees of insertion of the scope. The indicator 896 in state 896-4 shows flexing of the scope to indicate that the scope is in a bent state (or that the scope has started to bend) by representing the scope in a flexed form. In some embodiments, one or more reference user interface elements (e.g., reference planes as shown in FIG. 31B or the reference user interface element 888 shown in FIG. 31C) are displayed with the indicator 896. In some embodiments, the one or more reference user interface elements are displayed in accordance with a determination that the scope is bent (e.g., the one or more reference user interface elements are displayed only after the scope is bent and not before). In some embodiments, the one or more reference user interface elements cease to be displayed in accordance with a determination that the scope is straight (e.g., the one or more reference user interface elements are removed as the scope returns from a bent state to a straight state). In some embodiments, the one or more reference user interface elements are displayed at all times. In some embodiments, the one or more reference user interface elements are not shown.

In some embodiments, the indicator 880 or 896 is displayed in a picture-in-picture window, such as window 3102, 3104, or 3106 shown in FIG. 31E. In some embodiments, the picture-in-picture windows are placed in corners of the user interface 802. This reduces distraction to an operator (e.g., a surgeon).

In some embodiments, the picture-in-picture windows shown in FIG. 31E are moved away from the user interface 802 so that the picture-in-picture windows cease to overlap with the user interface 802 (e.g., the picture-in-picture windows, such as windows 3102, 3104, and 3106 move to locations below the user interface 802) as shown in FIG. 31F. In some embodiments, the user interface 802 is scaled (e.g., shrinks) and/or moved (e.g., the center of the user interface 802 moves upward) to provide space on the display device 800 for display of the windows away from the user interface 802 as shown in FIG. 31F.

In some embodiments, the visual representations that are illustrated in FIGS. 28A to 20D, 29A to 29D, 30A to 30F, and 31A-31F, or one or more portions thereof, are color coded. In some embodiments, at least a portion (e.g., the straight portion) of the indicator 896 may be color coded to indicate a percentage of insertion. For example, at least the straight portion of the indicator 896 has a first color, such as green, while the scope is inserted between 0% and 80%, a second color, such as yellow, while the scope is inserted between above 80% and 99%, and a third color, such as red, while the scope is in a state of 100% insertion. In some embodiments, at least a portion of the indicator 896 may be color coded to indicate a degree of flexure of the flexible end of the scope (e.g., at least a portion of the indicator 896 has a particular color, such as red, to indicate that the scope may not flex further, or that the scope is in a state of a maximum flexure). In some embodiments, the degree of insertion of the scope or the degree of flexure of the scope is indicated with color without displaying the entire indicator 896. For example, a color indicator, which does not have a shape of a scope, is used to indicate the degree of insertion of the scope or the degree of flexure of the scope. In some embodiments, the degree of insertion of the scope or the degree of flexure of the scope is indicated with an animation associated with the indicator 896. For example, the indicator 896 may be displayed to vibrate at least for a predefined time to indicate the degree of insertion of the scope or the degree of flexure of the scope (e.g., that the scope has reached a state of full insertion or full flexure).

In some embodiments, the visual representations that are illustrated in FIGS. 28A to 20D, 29A to 29D, 30A to 30F, and 31A-31F are provided with one or more audible signals (e.g., dedicated sounds, for example, a first sound indicating that the scope is fully inserted and a second sound distinct from the first sound indicating that the scope is fully flexed).

In some embodiments, the visual representations that are illustrated in FIGS. 28A to 28D, 29A to 29D, 30A to 30F, and 31A-31F are displayed when the surgeon activates a “camera clutch” mode. In some embodiments, the surgeon activates the “camera clutch” mode by pressing a camera pedal (e.g., foot pedal 244) of the surgeon console 240, or by activating a button on the console touchscreen display, or by an arm touchpoint at the bedside.

In some embodiments, the visual representations that are illustrated in FIGS. 28A to 28D, 29A to 29D, 30A to 30C, and 31A-31F are displayed in accordance with a determination (e.g., by processors 380) that the robotic medical system is operating a flexible scope (e.g., regardless of whether the flexible scope is operating in a particular drive mode).

As described above, FIGS. 28A to 28D, 29A to 29D, and 30A to 30C illustrate exemplary user interfaces. A person having ordinary skill in the art would understand that one or more processors (e.g., central processing unit, graphics processing unit, accelerated processing unit, application-specific integrated circuit, etc.) may be configured to generate electrical signals (e.g., video signals) so that one or more display devices can display the user interfaces illustrated in FIGS. 28A to 28D, 29A to 29D, and 30A to 30C.

### F. Exemplary Processes for Displaying Visual Indicators.

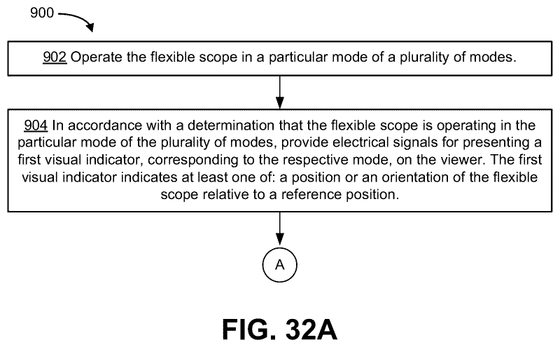

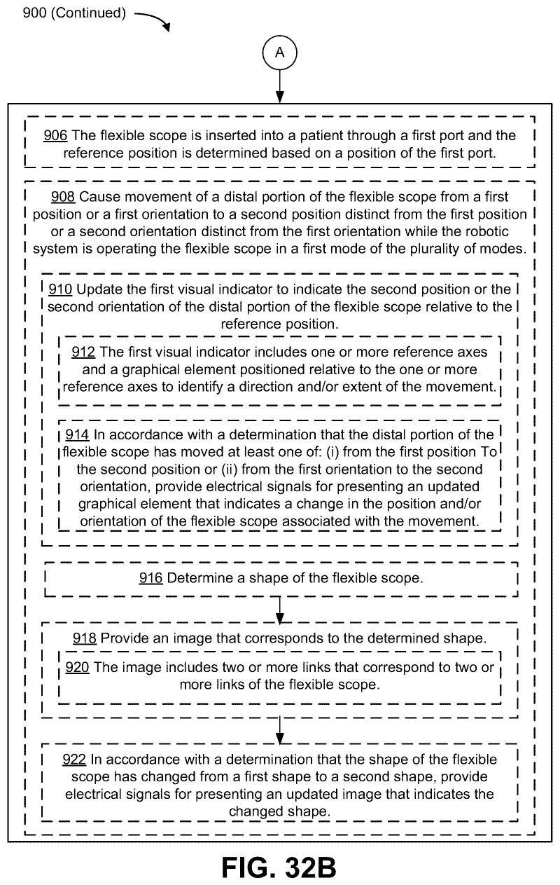

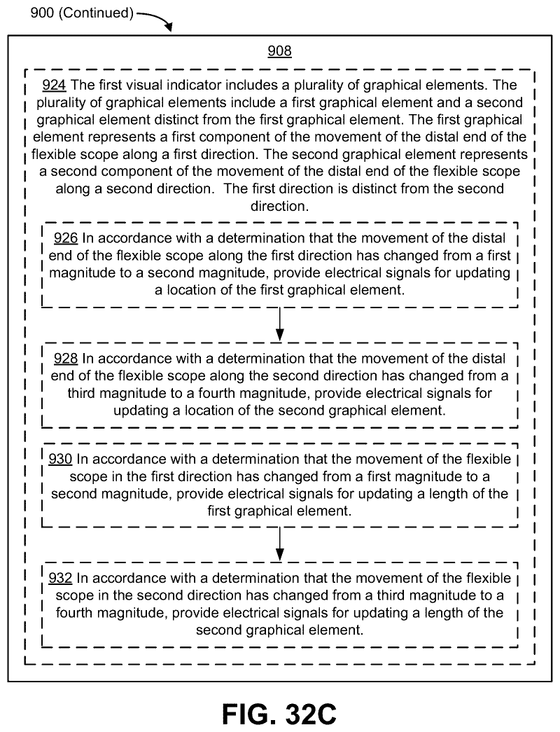

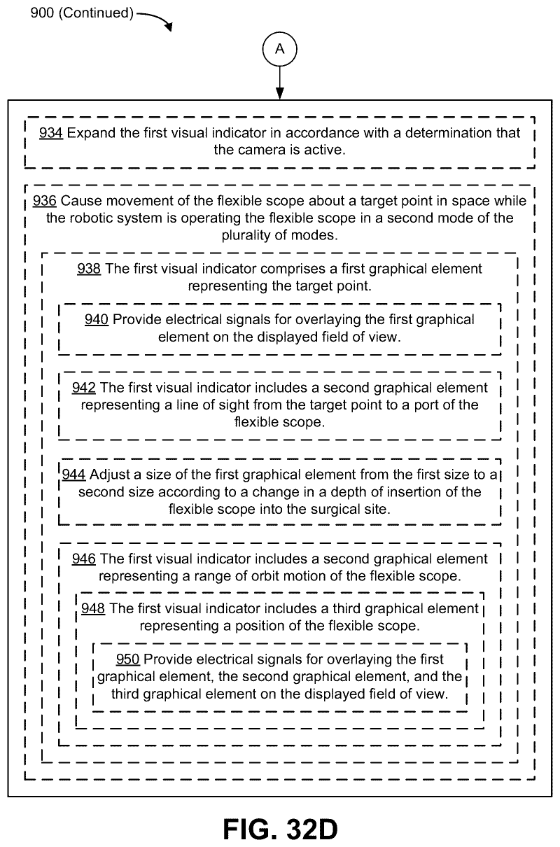

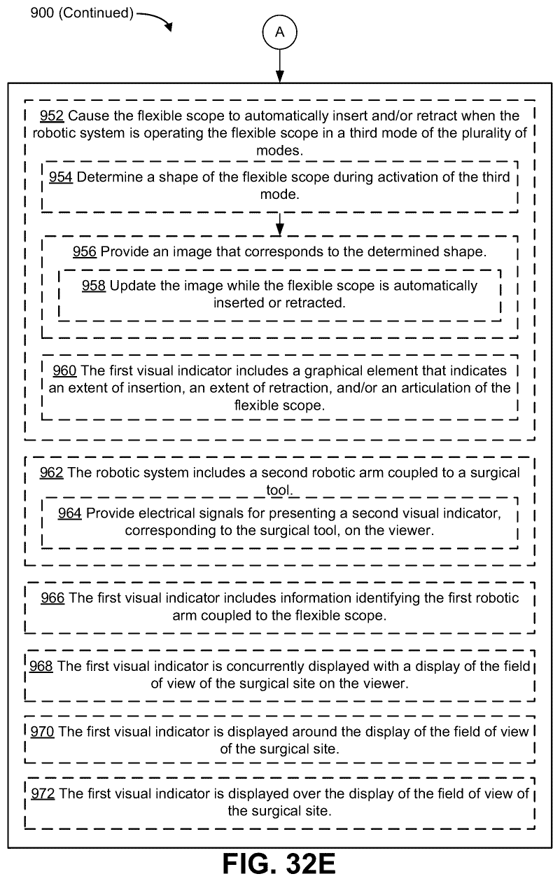

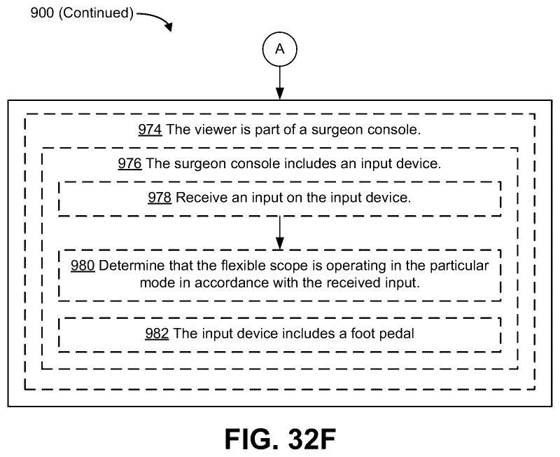

FIGS. 32A to 32F illustrate a flowchart diagram for a method 900 performed by one or more processors (e.g., processors 380) of a robotic system (e.g., the robotic medical system 200 as illustrated in FIGS. 21 and 22, a surgical robotic system, or a robotic surgery platform, etc.), in accordance with some embodiments. The surgical robotic system includes memory that stores instructions for execution by the one or more processors.

The robotic system includes a first robotic arm (e.g., a robotic manipulator) (e.g., the robotic arm 210-1 in FIG. 22) coupled to a flexible scope (e.g., flexible scope 720, such as a flexible laparoscope or a flexible endoscope).

The robotic system includes a viewer (e.g., display device 232 located on a tower 230, or display device 242 that is included with a physician console 240) for displaying a field of view of a surgical site derived from the flexible scope (e.g., the flexible scope is coupled to a camera at a distal end of the scope) (the flexible scope introduces new degrees of freedom, allowing a surgeon to control the camera in more ways than with a rigid scope).

The robotic system includes one or more processors (e.g., processor 380) and memory (e.g., memory 382) storing instructions for execution by the one or more processors.

The robotic system operates (902) the flexible scope in a particular mode (e.g., drive mode) of a plurality of modes (e.g., a respective (drive) mode of a plurality of (drive) modes). In some embodiments, the plurality of modes includes an articulation mode, an orbit mode, and/or an automatic insertion and retraction mode. In some embodiments, the plurality of modes also includes one or more additional modes.

The robotic system, in accordance with a determination (904) that the flexible scope is operating in the particular mode of the plurality of modes, provides electrical signals (e.g., video signals, such as analog video signals or digital video signals) for presenting a first visual indicator (e.g., indicator 806 in FIGS. 28A, 28B, and 28C, indicator 822 in FIG. 28D, indicator 832 in FIGS. 29A and 29B, indicator 844 in FIG. 29C), indicator 854 in FIG. 29D, indicator 860 in FIGS. 30A and 30B, or indicator 866 in FIG. 30C), corresponding to the respective mode, on the viewer. The first visual indicator indicates at least one of: a position (e.g., a first position) or an orientation (e.g., a first orientation) of the flexible scope (e.g., a position and/or orientation of a distal end of the flexible scope) relative to a reference position.

In some embodiments, the flexible scope is inserted (906) into a patient through a first port (e.g., port 608) and the reference position is determined based on (e.g., corresponds to) a position of the first port. For example, in some embodiments, the flexible scope is aligned with a line of sight from the cannula to the port when it is inserted through the port into the surgical site.

In some embodiments, the robotic system (e.g., via the one or more processors) causes (908) movement (e.g., an articulation movement, a tilt, a rotation, a pivot motion, a rotatory motion, etc.) of (e.g., with respect to, proximate to) a distal portion of the flexible scope (e.g., at a distal end 724 of the flexible scope 720, at a link 726 located at a distal portion of the flexible scope, at a joint 728 at a distal portion of the flexible scope, etc.) from a first position or a first orientation to a second position distinct from the first position or a second orientation distinct from the first orientation while the robotic system is operating the flexible scope in a first mode (e.g., articulation drive mode) of the plurality of modes.

For example, the movement of the distal portion of the flexible scope places the flexible scope into a second position (distinct from the first position) or a second orientation (distinct from the first orientation), and the first visual indicator is updated to indicate the second position or second orientation of the flexible scope (or the distal portion thereof).

In some embodiments, the movement of the distal end of the flexible scope comprises movement in an upward, downward, leftward, and/or rightward direction.

In some embodiments, the flexible scope includes a proximal end (e.g., intermediate portion 722) that remains stationary when the distal portion is caused to move from the first position or the first orientation to the second position or the second orientation.

In some embodiments, the robotic system updates (910) the first visual indicator to indicate the second position or the second orientation of the distal portion of the flexible scope relative to the reference position (e.g., relative to a neutral position of the flexible scope) (e.g., when the flexible scope is fully aligned (straight) with a line of sight from the cannula to the port). For example, in FIG. 28B, the indicator 806 is updated to indicate the second position or the second orientation of the distal portion of the flexible scope.

In some embodiments, the first visual indicator includes (912) one or more reference axes and a graphical element (e.g., a marker) positioned relative to the one or more reference axes to identify the direction and/or extent of the movement. For example, the indicator 806 includes reference axes 807 and a graphical element (e.g., marker) 808 positioned relative to the reference axes 807 to identify the direction and/or extent of the movement.

In some embodiments, the robotic system, in accordance with a determination (914) (e.g., automatically, in real-time) that the distal portion of the flexible scope has moved from a first position and/or a first orientation to a second position and/or a second orientation, provides (e.g., automatically, in real-time) electrical signals for presenting an updated graphical element (e.g., graphical element 811) that indicates a change in the position and/or orientation of the flexible scope associated with the movement.

In some embodiments, the robotic system determines (916) (e.g., in real time) a shape of the flexible scope. For example, the robotic system may determine the shape of the flexible scope from control signals provided to the flexible scope. The robotic system provides (918) an image (e.g., a rendered image, such as a 3-D rendered image) (e.g., representation 812) that corresponds to (e.g., indicates, identifies, or includes) the determined shape.

In some embodiments, the image includes (920) two or more links (e.g., links 814) that correspond to two or more links (e.g., links 726) of the flexible scope.

In some embodiments, the robotic system, in accordance with a determination (922) (e.g., in real time) that the shape of the flexible scope has changed from a first shape to a second shape (e.g., due to a change in the direction and/or degree of the articulation), providing electrical signals for presenting an updated image (e.g., representation 813) that indicates the changed shape.

In some embodiments, the first visual indicator includes (924) a plurality of graphical elements (e.g., two bars (white portions of the bars), rectangles, ellipses, etc.). The plurality of graphical elements includes a first graphical element (e.g., graphical element 828-1) (e.g., a bar, a rectangle, a line, an ellipse, etc.) and a second graphical element (e.g., graphical element 828-2) (e.g., a bar, a rectangle, a line, an ellipse, etc.) distinct from the first graphical element. The first graphical element represents a first component (e.g., a direction and/or extent) of the movement of the distal end of the flexible scope along a first direction (e.g., left or right). The second graphical element represents a second component (e.g., a direction and/or extent) of the movement of the distal end of the flexible scope along a second direction (e.g., upward or downward). The first direction is distinct from the second direction (e.g., the second direction is perpendicular to the first direction, the first and second directions are not parallel to each other).

In some embodiments, the robotic system, in accordance with a determination (926) (e.g., in real-time) that the movement of the distal end of the flexible scope along the first direction has changed from a first magnitude to a second magnitude, provides electrical signals for updating a location of the first graphical element. In some embodiments, the robotic system, in accordance with a determination (928) (e.g., in real-time) that the movement of the distal end of the flexible scope along the second direction has changed from a third magnitude to a fourth magnitude, providing electrical signals for updating a location of the second graphical element.

In some embodiments, the robotic system, in accordance with a determination (930) (e.g., in real-time) that the movement of the flexible scope in the first direction (e.g., left or right) has changed from a first magnitude to a second magnitude, provides electrical signals for updating a length (e.g., length 830-1) of the first graphical element (e.g., graphical element 828-1). In some embodiments, the robotic system, in accordance with a determination (932) (e.g., in real-time) that the movement of the flexible scope in the second direction (e.g., up or down) has changed from a third magnitude to a fourth magnitude, provides electrical signals for updating a length (e.g., length 830-2) of the second graphical element (e.g., graphical element 828-2).

In some embodiments, the field of view of the surgical site is derived from a camera coupled to the flexible scope (e.g., at a distal portion of the scope). The robotic system expands (934) the first visual indicator (e.g., the scope tab) in accordance with a determination that the camera is active. For example, as illustrated in FIG. 28A, in some embodiments, the indicator 806 may have an initial size that is similar to the indicators 804. The size of the indicator 806 expands when the camera coupled to the flexible scope 720 is active.

In some embodiments, the robotic system causes (936) movement of the flexible scope about a target point in space (e.g., target point 734) while the robotic system is operating the flexible scope in a second mode (e.g., an orbit drive mode) of the plurality of modes (e.g., an orbit point) (e.g., an orbit drive mode).

In some embodiments, the target point is predefined (e.g., selected) by the surgeon. In some embodiments, the target point is predefined by the robotic system. In some embodiments, the target point is randomly selected by the robotic system. In some embodiments, the target point corresponds to the center of the field of view of the camera at the time the orbit drive mode is activated. In some embodiments, the target point is located off the center of the field of view of the camera.

In some embodiments, the first visual indicator comprises (938) a first graphical element (e.g., graphical element 834) (e.g., a closed circle, an open circle, a sphere, a cross) representing the target point.

In some embodiments, the robotic system provides (940) electrical signals for overlaying the first graphical element on the displayed field of view (e.g., superimposing the first point over the displayed field of view of the surgical site). For example, in FIG. 29A, the graphical element 834 is overlaid on the representation 840 of the field of view.

In some embodiments, the first visual indicator includes (942) a second graphical element (e.g., graphical element 836) (e.g., a straight line, a solid line, a dashed line) representing a (e.g., direct) line of sight from the target point to a port of the flexible scope.

In some embodiments, the first visual indicator includes a text element (e.g., element 838) (e.g., “Scope port”) that indicates a direction of the scope port.

In some embodiments, when the port, the camera, and the target point are aligned (meaning that the port is directly behind the camera), the second graphical element converges/merges with the first graphical element.

In some embodiments, the first graphical element (e.g., graphical element 834) has (e.g., is characterized by) a first size (e.g., a diameter, a length, a width, an area, etc.). The robotic system adjusts (944) (e.g., changes, varies, etc.) a size of the first graphical element (e.g., in real time) from the first size to a second size according to a change in a depth of insertion of the flexible scope into the surgical site (e.g., the graphical element 834 in FIGS. 29A and 29B).

In some embodiments, the first size is a predefined by one or more processors (e.g., processor 380) of the robotic system.

In some embodiments, the first size indicates a distance from the flexible scope to a target organ.

In some embodiments, the first size indicates an insertion distance of the scope (e.g., how far the scope is inserted into the body). For example, a larger size indicates the scope is inserted further into the body whereas a smaller size indicates the scope is closer to the surface.

In some embodiments, the size of the first graphical element increases in accordance with a determination that the depth of insertion has increased (e.g., the flexible scope is inserted further into the surgical site). In some embodiments, the size of the first graphical element decreases in accordance with a determination that the depth of insertion has decreased (e.g., the flexible scope is moving further away from the surgical site).

In some embodiments, the first visual indicator (e.g., indicator 844) includes (946) a second graphical element (e.g., graphical element 846) (e.g., a sphere, a globe, etc.) representing a range of orbit motion of the flexible scope.

In some embodiments, the first visual indicator (e.g., indicator 844) includes (948) a third graphical element (e.g., graphical element 850) (e.g., closed circle, a marker, such as a “x”, etc.) representing a (e.g., real-time, current) position of the flexible scope.

In some embodiments, the robotic system provides (950) electrical signals for overlaying the first graphical element, the second graphical element, and the third graphical element on the displayed field of view (e.g., the graphical elements 846, 848, and 850 in FIG. 29C).

In some embodiments, the first visual indicator (e.g., indicator 854) includes one or more axes (e.g., three axes, such as axes 856) that represent an insertion direction (or rotation) of the flexible scope relative to the one or more axes (e.g., the axes 856 in FIG. 29D). In some embodiments, the one or more axes are located adjacent to a top, bottom, left or right edge of the user interface 802. In some embodiments, the one or more axes are located to a corner of the user interface 802.

In some embodiments, the robotic system causes (952) the flexible scope to automatically (e.g., without manual force applied on the flexible scope) insert (e.g., from a cannula of the first robotic arm to the surgical site, from the port into the surgical site, etc.) and/or (automatically) retract (e.g., from the surgical site to the port, from the surgical site to the cannula of the first robotic arm, etc.) when the robotic system is operating the flexible scope in a third mode (e.g., automatic insertion and retraction mode) of the plurality of modes.

In some embodiments, the robotic system determines (954) (e.g., in real time) a shape of the flexible scope during activation of the third mode (or during the automatic insertion or the automatic retraction). For example, the robotic system may determine the shape of the flexible scope based on control signals provided to the flexible scope. Alternatively, the robotic system may determine the shape of the flexible scope based on signals indicating the positions or states of respective joints of the flexible scope. The robotic system provides (956) an image (e.g., a rendered image, such as a 3-D rendered image) that corresponds to the determined shape (e.g., the graphical element 861 in FIGS. 30A and 30B).

In some embodiments, the robotic system updates (958) (e.g., automatically, in real-time) the image while the flexible scope is automatically inserted or retracted (e.g., the graphical element 862 in FIG. 30B).

In some embodiments, the first visual indicator (e.g., indicator 866) includes (960) a graphical element (e.g., graphical element 868) (e.g., a bar, a shading, etc.) that indicates an extent of insertion (e.g., fully inserted, partially inserted, etc.) and/or an extent of retraction (e.g., fully retracted, partially retracted, etc.). In some embodiments, the first visual indicator (e.g., indicator 866) includes a graphical element (e.g., graphical element 872) that indicates an articulation (e.g., straight, articulated, etc.) of the flexible scope.

In some embodiments, the robotic system includes (962) a second robotic arm (e.g., a robotic manipulator) (e.g., the robotic arm 210-2) coupled to a surgical tool (e.g., medical instrument 212 in FIGS. 21 and 24A, surgical tools 602 in FIG. 25, and surgical tool 704 and 708 in FIGS. 26A and 26B).

In some embodiments, the robotic system provides (964) electrical signals for presenting a second visual indicator (e.g., indicator 804), corresponding to the surgical tool, on the viewer. In some embodiments, the second visual indicator identifies medical instruments (e.g., surgical) at the surgical site. In some embodiments, the second visual indicator 804 includes an association between a robotic arm and a respective medical instrument to which it is coupled. For example, in FIG. 28A, the indicator 804-2 includes a number “2” indicating that Tool B is coupled to a second robotic arm of the robotic medical system.

In some embodiments, the first visual indicator includes (966) information (e.g., robotic arm number) identifying the first robotic arm coupled to the flexible scope. For example, in FIGS. 28A, 28B, and 28C, the vindicator 806 includes the number “3” identifying the robotic arm (e.g., robotic arm 3) coupled to the flexible scope.

In some embodiments, the first visual indicator is concurrently displayed (968) with a display of the field of view of the surgical site on the viewer (e.g., the indicators in FIGS. 28A to 28D, FIGS. 29A to 29D, and FIGS. 30A to 30C).

In some embodiments, the first visual indicator is displayed (970) around the display of the field of view of the surgical site (e.g., along a periphery of the display of the field of view of the surgical site) (e.g., the indicators in FIGS. 28A to 28D, FIG. 29D, and FIG. 30C).

In some embodiments, the first visual indicator is displayed (972) over the display of the field of view of the surgical site (e.g., the indicators in FIGS. 28A to 28D, FIGS. 29A to 29D, and FIGS. 30A to 30C).

In some embodiments, the viewer is (974) part of a surgeon console (e.g., the display device 242 is included in the physician console 240).

In some embodiments, the surgeon console includes (976) an input device (e.g., a user interface of the display device 242, input devices such as buttons, switches, touch-sensitive surfaces, or gimbals, a foot pedal 244, etc.). The robotic system receives (978) an input on the input device. The robotic system determines (980) that the flexible scope is operating in the particular mode in accordance with the received input.

In some embodiments, the input device includes (982) a foot pedal (e.g., foot pedal 244).

In some embodiments, the first visual indicator includes a graphical representation of the flexible scope in a non-primary graphical projection.

In some embodiments, the robotic system displays one or more reference user interface elements in accordance with a determination that the flexible scope is flexed.

In some embodiments, the robotic system ceases to display the one or more reference user interface elements in accordance with a determination that the flexible scope is straight.

### 3. Implementing Systems and Terminology.

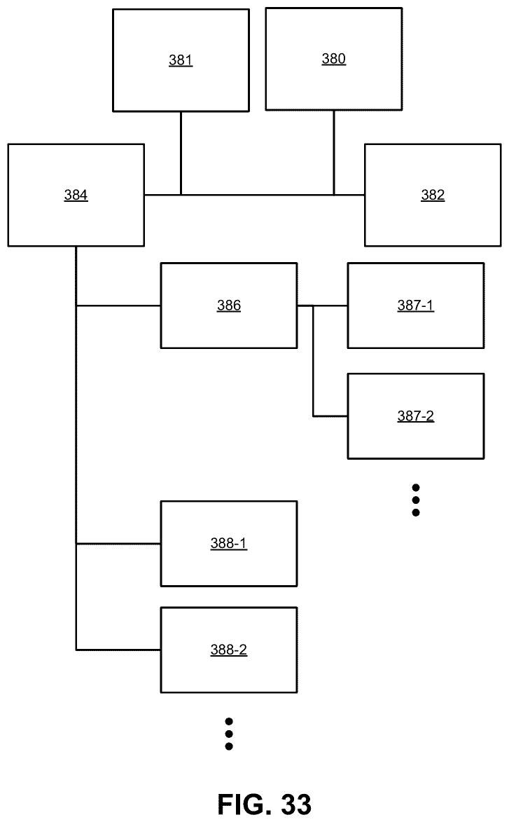

FIG. 33 is a schematic diagram illustrating electronic components of a medical robotic system (e.g., a surgical robotic system) in accordance with some embodiments.

The robotic medical system (e.g., surgical robotic system) includes one or more processors 380, which are in communication with a computer-readable storage medium 382 (e.g., computer memory devices, such as random-access memory, read-only memory, static random-access memory, and non-volatile memory, and other storage devices, such as a hard drive, an optical disk, a magnetic tape recording, or any combination thereof) storing instructions for performing any methods described herein (e.g., operations described with respect to FIGS. 25, 26A, 26B, 27A to 27D, 28A to 28D, 29A to 29D, 30A to 30C, and 32A to 32F). The one or more processors 380 are also in communication with an input/output controller 384 (via a system bus or any suitable electrical circuit). The input/output controller 384 receives sensor data from one or more sensors 388-1, 388-2, etc., and relays the sensor data to the one or more processors 380. The input/output controller 384 also receives instructions and/or data from the one or more processors 380 and relays the instructions and/or data to one or more actuators, such as first motors 387-1 and 387-2, etc. In some embodiments, the input/output controller 384 is coupled to one or more actuator controllers 386 and provides instructions and/or data to at least a subset of the one or more actuator controllers 386, which, in turn, provide control signals to selected actuators. In some embodiments, the one or more actuator controller 386 are integrated with the input/output controller 384 and the input/output controller 384 provides control signals directly to the one or more actuators 387 (without a separate actuator controller). Although FIG. 30 shows that there is one actuator controller 386 (e.g., one actuator controller for the entire medical robotic system; in some embodiments, additional actuator controllers may be used (e.g., one actuator controller for each actuator, etc.). In some embodiments, the one or more processors 380 are in communication with one or more displays 381 for displaying information as described herein.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The functions for determining whether a tool is within or outside a surgical field of view provided by a camera or scope and rendering one or more indicators representing positions or directions of one or more medical tools described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

As used herein, the term “exemplary” means “serving as an example, instance, or illustration,” and does not necessarily indicate any preference or superiority of the example over any other configurations or implementations.

As used herein, the term “and/or” encompasses any combination of listed elements. For example, “A, B, and/or C” includes the following sets of elements: A only, B only, C only, A and B without C, A and C without B, B and C without A, and a combination of all three elements, A, B, and C.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the embodiments shown herein but is to be accorded the widest scope consistent with the principles and novel features disclosed herein.

What is claimed is:

1. A robotic system, comprising:
a first robotic arm coupled to a flexible scope;
a viewer for displaying a field of view of a surgical site derived from the flexible scope;
one or more processors; and
a memory configured to store instructions for execution by the one or more processors, the stored instructions including instructions for:
operating the flexible scope in a particular mode of a plurality of modes; and
in accordance with a determination that the flexible scope is operating in the particular mode of the plurality of modes, providing electrical signals for presenting a first visual indicator, corresponding to the respective mode, on the viewer, the first visual indicator indicating at least one of: a position or an orientation of the flexible scope relative to a reference position.

2. The robotic system of claim 1, wherein the flexible scope is inserted into a patient through a first port and the reference position is determined based on a position of the first port.

3. The robotic system of claim 1, wherein:
the robotic system causes movement of a distal portion of the flexible scope from a first position or a first orientation to a second position distinct from the first position or a second orientation distinct from the first orientation while the robotic system is operating the flexible scope in a first mode of the plurality of modes, and
the stored instructions include instructions for updating the first visual indicator to indicate the second position or the second orientation of the distal portion of the flexible scope relative to the reference position.

4. The robotic system of claim 3, wherein the first visual indicator includes one or more reference axes and a graphical element positioned relative to the one or more reference axes to identify at least one of a direction or an extent of the movement.

5. The robotic system of claim 3, wherein the stored instructions also include instructions for:
in accordance with a determination that the distal portion of the flexible scope has moved at least one of: (i) from the first position to the second position or (ii) from the first orientation to the second orientation, providing electrical signals for presenting an updated graphical element that indicates a change in at least one of the position or orientation of the flexible scope associated with the movement.

6. The robotic system of claim 3, wherein the first visual indicator includes a plurality of graphical elements, the plurality of graphical elements including a first graphical element and a second graphical element distinct from the first graphical element, wherein:
the first graphical element represents a first component of the movement of a distal end of the flexible scope along a first direction; and
the second graphical element represents a second component of the movement of the distal end of the flexible scope along a second direction, wherein the first direction is distinct from the second direction.

7. The robotic system of claim 6, wherein the stored instructions also include instructions for:
in accordance with a determination that the movement of the distal end of the flexible scope along the first direction has changed from a first magnitude to a second magnitude, providing electrical signals for updating a location of the first graphical element; and
in accordance with a determination that the movement of the distal end of the flexible scope along the second direction has changed from a third magnitude to a fourth magnitude, providing electrical signals for updating a location of the second graphical element.

8. The robotic system of claim 6, wherein the stored instructions also include instructions for:
in accordance with a determination that the movement of the flexible scope in the first direction has changed from a first magnitude to a second magnitude, providing electrical signals for updating a length of the first graphical element; and
in accordance with a determination that the movement of the flexible scope in the second direction has changed from a third magnitude to a fourth magnitude, providing electrical signals for updating a length of the second graphical element.

9. The robotic system of claim 1, wherein:
the field of view of the surgical site is derived from a camera coupled to the flexible scope; and
the stored instructions also include instructions for expanding the first visual indicator in accordance with a determination that the camera is active.

10. The robotic system of claim 1, wherein the robotic system causes movement of the flexible scope about a target point in space while the robotic system is operating the flexible scope in a second mode of the plurality of modes.

11. The robotic system of claim 10, wherein the first visual indicator comprises a first graphical element representing the target point, wherein the stored instructions also include instructions for providing electrical signals for overlaying the first graphical element on the field of view, wherein the first visual indicator includes a second graphical element representing a line of sight from the target point to a port of the flexible scope.

12. The robotic system of claim 11, wherein:
the first graphical element has a first size; and
the stored instructions also include instructions for:
adjusting a size of the first graphical element from the first size to a second size according to a change in a depth of insertion of the flexible scope into the surgical site.

13. The robotic system of claim 11, wherein the first visual indicator includes a second graphical element representing a range of orbit motion of the flexible scope.

14. The robotic system of claim 1, wherein the first visual indicator includes:
one or more axes; and
a graphical element that represents an insertion direction of the flexible scope relative to the one or more axes.

15. The robotic system of claim 1, wherein the robotic system causes the flexible scope to automatically insert and/or retract when the robotic system is operating the flexible scope in a third mode of the plurality of modes, wherein the stored instructions also include instructions for:
determining a shape of the flexible scope during activation of the third mode; and
providing an image that corresponds to the determined shape; and
updating the image while the flexible scope is automatically inserted or retracted.

16. The robotic system of claim 1, further comprising:
a second robotic arm coupled to a surgical tool, wherein the stored instructions also include instructions for providing electrical signals for presenting a second visual indicator, corresponding to the surgical tool, on the viewer.

17. The robotic system of claim 1, wherein the viewer is part of a surgeon console, wherein:
the surgeon console includes a foot pedal; and
the stored instructions include instructions for:
receiving an input on the foot pedal; and
determining that the flexible scope is operating in the particular mode in accordance with the received input.

18. The robotic system of claim 1, wherein the first visual indicator includes a graphical representation of the flexible scope in a graphical projection.

19. The robotic system of claim 18, wherein the stored instructions include instructions for displaying one or more reference user interface elements in accordance with a determination that the flexible scope is flexed.

20. The robotic system of claim 19, wherein the stored instructions include instructions for ceasing to display the one or more reference user interface elements in accordance with a determination that the flexible scope is straight.
